(19) United States
(12) Patent Application Publication
(10) Pub. No.: US 2007/0282301 A1
(43) Pub. Date: Dec. 6, 2007

(54) Dilatation Balloon Catheter Including External Means For Endoluminal Therapy And For Drug Activation

(76) Inventors: Victor A. Segalescu, Haifa (IL); Gabriel Bartal, RaAnana (IL)

Correspondence Address: Martin D. Moynihan;Prtsi Inc., P.O. Box 16446, Arlingon, VA, 22215, US

(21) Appl. No.: 10/590,656

(22) PCT Filed: Feb. 27, 2005
(86) PCT No.: PCT/IL05/00234

§ 371 Date: Jun. 25, 2007

Related U.S. Application Data
(60) Provisional application No. 60/547,447, filed on Feb. 26, 2004.

(51) Int. Cl. A61M 31/00; A61B 1/00

(52) U.S. Cl. 604/509; 600/101

(57) ABSTRACT

An inflatable device to be used in treatment of stenotic lesions includes external optical fibers with a segment capable of emitting substantially uniform radial energy. The design of the optical fibers ensures a high optical coupling factor and a high efficiency in delivering light-energy to the adjacent tissue volume. A light source is connected to the optical fibers during the inflation of the device. The parameters of the light source: wavelength, waveform and intensity and the design of the optical fibers ensure that the light-tissue interaction depth is within a thin layer of stenotic lesion without inducing irreversible damage to the vessel wall. During the inflation of the device, the external optical fibers pressure the same tissue volume that interacted with the radial emitted light-energy. The confined radial light energy and the mechanical effects induced by the optical fibers create an opto-mechanical effect that facilitates the dilatation of stenotic lesions and reduces the risk of restenosis. The external optical fibers can also facilitate endoluminal drug activation, wherein light activated drugs are used to prevent restenosis or to treat cardiovascular, system, benign and malignant stenosis and other diseases. Various combinations of optical fibers with different capabilities can be used in the same device. The device can be used in treatment of stenosis in the vascular system and in non-vascular systems (such as the urinary or biliary systems). The design of the device ensures that the attachment of the external optical fibers does not obstruct the advancement and maneuvering of the device through tortuous anatomical structures such as stenotic or partially occluded blood vessels.

### FIELD OF THE INVENTION

The present invention relates to devices and methods that facilitate percutaneous endoluminal therapy of blood vessels or other anatomical structures. More particularly, the present invention relates to devices that are capable of dilating a stenotic vessel while reducing the risk of restenosis after the procedure.

The teachings of U.S. Provisional Patent Application No. 60/547,447, filed 26 Feb. 2004 are incorporated herein by reference.

### BACKGROUND OF THE INVENTION

Percutaneous transluminal angioplasty (PTA) using a dilatation balloon catheter remains the most effective and widely used interventional endoluminal technique in treatment of stenosis in the vascular system and in non-vascular systems (such as urinary or biliary systems). Percutaneous transluminal coronary angioplasty is further referred as PTCA. Inflation of an angioplasty balloon in a stenotic artery, for example, causes splitting or dissection of the artherosclerotic plaque and adjacent intima, and stretching of the muscularis media, thus increasing the cross-sectional area of the artery. After the intervention, the intima re-endothelializes as part of the remodeling process. Three of the main obstacles faced by conventional PTA in cardiovascular applications are:
Difficulty of dilating rigid, elastic or diffusely diseased segments;
Difficulty of preventing or reversing abrupt closure of the dilated segment;
Virtually inability to reduce below 30%-40% the incidence of restenosis after successful expansion of the blood vessel lumen without using additional therapy means such as stenting.

In order to improve clinical results in cardiovascular applications, the dilatation of a blood vessel lumen by inflating a dilatation balloon catheter is followed by stenting in more than 90% of the currently performed PTA/PTCA procedures.

Over the years several adjacent mechanisms were added to the original PTA/PTCA aperture in order to improve the dilatation mechanism of the basic dilatation balloon catheter. These mechanisms include (among others):
Addition of very small metal blades on the perimeter of the balloon body in order to create during the inflation small cuts in the atherosclerotic plaque—U.S. Pat. No. 5,320,634 (Virgil et all.). These cuts are intended to assist during the splitting and dissection process. The length of such blades is significantly limited due to maneuvering constraints and safety issues.
Addition of metal wire/s on the perimeter of the balloon body in order to apply a concentrated stress on the atherosclerotic plaque and on the blood vessel wall during dilatation—U.S. Pat. No. 6,394,995 (Solar et all.).

References have been made in the literature regarding the usage of lasers for endoluminal therapy purposes. Most of the references are related to laser delivery means capable of emitting forward focused light directed to open an occluded vessel lumen. Other described laser delivery means—see for example, U.S. Pat. No. 5,466,234, U.S. Pat. No. 5,741,246, U.S. Pat. No. 5,891,082, U.S. Pat. No. 6,159,236 and U.S. Pat. No. 6,485,485 use lateral transmission of light from the inside of the balloon body through a transparent balloon material for healing purposes after the inflation process. Spears et all. describe in U.S. Pat. Nos. 5,226,430 and 5,019,075 the option of heating the region surrounding a balloon used for PTCA by means within the balloon or within the skin of the balloon in order to fuse together fragmented segments of tissue. In U.S. Pat. No. 5,624,433, Radisch describes the usage of grooved laser rods for using light incision while dilating a balloon. The grooved laser-rods described by Radisch, if used, would cause severe collateral thermal damage due to their inherent low efficiency and therefore would lead to an unwanted high-level of restenosis.

Alternative referenced therapy methods employ ionizing radiation delivered to the required stenotic lesion after a PTA procedure (also referenced as brachytherapy means).

An alternative method for decreasing the restenosis rate is the usage of drugs that prevent cell proliferation and assist in the healing process. The most prominent device based on this therapy effect is the new generation of “Drug-Eluting Stents” that has been developed by various companies. Drug Eluting Stents are stents coated with special drugs that are slowly released in the vessel wall in order to prevent cell proliferation (after stenting) thus preventing possible in-stent restenosis. Clinical results show that after usage of Drug-Eluting Stents the rate of in-stent restenosis is much lower, but can still be above 8% depending on the type of patients and lesions (diabetic/non-diabetic, etc). The efficacy of Drug Eluting Stents, however, has been established mainly in coronary applications, whereas in other vessels the treatment of stenotic lesions still requires alternative solutions. For stenotic lesions in other anatomic locations, the current restenosis rate is in the range of 10% to 30% of cases even after placement of a balloon expandable or self-expandable regular stent (and in some anatomic areas the restenosis rate is even higher). In such cases, it is necessary to treat the stenotic area again, and PTA/PTCA is often chosen as the preferred way of re-canalizing the restenotic segment.

Alternative methods for delivering and activating drugs that are efficient in preventing restenosis are developed/disclosed by several other companies. One of the proposed methods is Photodynamic Therapy (PDT) wherein light activated drugs are used to locally and selectively prevent or treat restenosis. U.S. Pat. Nos. 5,997,570 and 6,159,236 describe balloon catheters with light sources inside the balloon for performing Photodynamic Therapy.

It is the purpose of the current invention to improve existing PTA/PTCA techniques and provide to the basic PTA apparatus additional means and capabilities that will improve the clinical outcome and long-term results of PTA.

It is also the purpose of the current invention to disclose an inflatable device that includes optical means capable of interacting with a thin layer of stenotic lesion without inducing irreversible damage to the vessel wall. Moreover, these optical or means can be selectively activated during the dilatation of the stenotic lesion, without obstructing the advancement and maneuvering of the device through tortuous anatomical structures such as stenotic or partially occluded blood vessels. Particularly, it is the purpose of the current invention to disclose the usage of optical fibers that are externally attached onto the balloon body, wherein the optical fibers have a distal segment capable of radial emitting light energy to the adjacent tissue, and wherein the external optical fibers facilitate a smooth dilatation of a stenotic lesion through a combination of light energy and mechanical means by creating longitudinal microscopic cuts or cracks around the distal segment without damaging the vessel wall and without inducing undesirable collateral thermal damage to the vessel tissue.

It is also the purpose of the present invention to describe methods and apparatus for decreasing the pressure required during the dilatation of the balloon in order to reduce the stress to the vessel wall that is of particular importance in reducing restenosis.

It is also the purpose of the current invention to disclose efficient means for activating photosensitive drugs during an angioplasty procedure. All the means and methods disclosed in the current invention are meant to overcome the clinical limitations of existing PTA/PTCA procedure.

The disclosed endoluminal therapy apparatus and methods can be used in treatment of stenosis, re-stenosis and in-stent restenosis, and are particularly effective in preventing acute and late restenosis. The apparatus and methods disclosed can preclude the usage of endoluminal stents or can be used as an adjunct to stenting. The apparatus and methods described are equally useful in assisting primary-stenting diminishing clinical complications that might be associated with such a procedure.

The current invention is also applicable to other clinical applications such as treatment of other various cardiovascular, and nonvascular diseases.

### SUMMARY OF THE INVENTION

Throughout the document light means an electromagnetic wave of various wavelength from 0.2 micron (UV) and up to 12.0 micron (IR). Throughout the document light energy delivering elements refer to optical fibers, hollow glass waveguides, photonic crystal fibers or other light-conductive means that may be used to deliver light or light energy to a required position in the body in relation to the present invention. Throughout the document a light source refers to any possible means of generating light or light energy, such as solid-state lasers, diode-lasers, gas-lasers, fiber-lasers, etc.

The present invention discloses apparatus and methods for treating a stenotic lesion in a vessel or similar clinical conditions. In a first disclosed embodiment a dilatation balloon catheter includes optical fibers externally attached onto the balloon body, wherein the optical fibers have a distal segment capable of emitting energy in a radial direction. The chosen optical design, the wavelength, the waveform and the intensity level of the light source enable to achieve a light-tissue interaction depth between the radial emitted energy and the plaque/tissue in a preferred range between 1 to 120 microns, without inducing thermal damage to tissue beyond this volume. The radial energy emission is activated during the dilatation of the balloon body while the dilatation of the balloon constantly brings the external radiating means in contact with the stenotic plaque or other type of tissue. The emission is selective and can be activated only at the beginning of the dilatation process or during the entire dilatation process. The emission is ceased (automatically, semi-automatically or manually) when the dilatation balloon has reached a certain size. The emission can optionally be matched (automatically, semi-automatically or manually) to the type of tissue proximal to the emitting means based on available information regarding the type of tissue. The dilatation balloon catheter also includes means of exerting focused pressure on, the same volume of tissue that interacts with the energy. The resulting opto-mechanical or thermo-mechanical effects cause removal or cracking or weakening of segments of microscopic width in the plaque or in the adjacent tissue, without inducing any thermal damage to the vessel tissue. The dilatation balloon catheter is preferably made from semi-compliant or non-compliant material, and its size is selected to dilate a stenotic lesion at lower pressure typically between 2 to 6 atmospheres, and preferably between 2 to 4 atmospheres. Preferably, the balloon will have a lower pressure range where it acts as a semi-compliant balloon and an upper pressure range, where it acts as a non-compliant balloon, in order to ensure that there is a pressure-range where the balloon inflation can benefit from the described opto-mechanical or thermo-mechanical effects induced by the radiating means, and at the same time there is no danger of over-stretching or inducing unwanted thermal or other damage to the vessel wall.

The inflation rate is gradual and matched to the effect induced by the radial emitting means and/or to the opto-mechanical or thermo-mechanical effects. The gradual inflation is preferably automatic or semi-automatic and correlated to the size and material from which the balloon is manufactured, to the change in the size and to the pressure level.

The first embodiment of the current invention discloses a dilatation balloon catheter with 1 to 4 optical fibers disposed longitudinally on the balloon body each optical fiber having a distal section, capable of emitting radial light-energy in at least one radial direction. The mechanical attachment of the optical fibers on the catheter ensures that the distal radial emitting section remains on the same position on the balloon body regardless of the inflation/deflation stage of the balloon and ensure that it remains in contact to the vessel wall and/or the stenotic tissue that may be covering it at all the stages during the inflation of the balloon. The mechanical attachment of the optical fibers also ensures the proper deployment and re-wrapping of the optical fibers during the inflation/deflation of the dilatation balloon. Before, during or after the dilatation of the dilatation balloon catheter, the externally attached optical fibers emit light energy through the distal section in a radial direction, where the radial emission pattern is substantially uniform along the entire emitting section. The design of the optical fibers ensures a high level of efficiency, in order to minimize the amount of heat dissipated to adjacent tissue, to minimize the size of the laser source and to minimize the required energy level through the fibers, therefore reducing the potential damage to the fibers. The selected wavelength, light waveform and intensity ensure that the interaction between the radial emitted light-energy and the plaque/tissue occurs only within a small volume of tissue adjacent to the emitting section without inducing thermal damage to the vessel wall. The interaction depth is typically selected between 1 to 50 microns (although other ranges can be selected per application), without inducing thermal damage to tissue beyond this volume. Optionally, the wavelength is selected according to specific cromophores in the tissue, or specific cromophores are induced in the plaque or adjacent tissue in order to increase the absorption coefficient at a selected wavelength. During the inflation of the balloon, the externally attached optical fibers pressure the exact longitudinal microscopic region that interacted with the radial confined light-energy delivery. Therefore, during the inflation process, the radial emitting section induces combined opto-mechanical effects into the adjacent tissue without causing collateral thermal damage, and facilitates the splitting of the atherosclerotic plaque or thrombosis.

For optimal, clinical results, the rate of increasing the pressure within the dilatation balloon has to be matched to the opto-mechanical effect induced by the external light delivery means (by methods disclosed in the present invention) ensuring that the dilatation of the vessel wall is made at a minimal pressure, and taking full advantage of the microscopic longitudinal opto-mechanical effects. As taught in the present invention, the material, the design and the size of the dilatation balloon body are selected such that it reaches the required diameter at significantly lower pressures than usual dilatation balloons (based on the fact that the dilatation process is assisted by the opto-mechanical effect created by the external light-delivery means).

Other embodiments of the current invention disclose alternative means of inducing microscopic effects capable of assisting the dilatation of a stenotic area based on alternative combinations of light-energy delivering and mechanical means.

The light-energy delivery means externally attached to the dilatation balloon catheter as disclosed in the above embodiments have additional clinical advantages due to their increased maneuverability inside the vessel lumen, and due to the capability of using virtually any length of the therapeutic length, as opposed to safety constraints on the lengths of possible alternative sharpened structures—see for example the atherotomes described by Virgil at all in U.S. Pat. No. 5,320,634.

Other embodiments of the current invention disclose apparatus and methods of using the light energy means externally attached to the dilatation balloon catheter for controlled delivery and activation of photosensitive drugs. In cardiovascular applications, for example, the external light delivery means can emit radial light energy that activates light sensitive drugs during and/or after the inflation of the dilatation balloon catheter and remodeling of the blood vessel wall. The activation of the light sensitive drugs that are effective in preventing or treating restenosis can be used as stand-alone therapy or by using the same light energy means and light source can be adjacent means to the opto-mechanical effect described in the first embodiment that creates; cuts or cracks in; the stenotic tissue and assists the dilatation process.

According to the present invention the same dilatation balloon catheter including light energy delivering elements and the same light source can be used to achieve multiple therapy effects based on variations of wavelength, duration or energy level of the delivered light energy.

Additional embodiments of the current invention disclose alternative apparatus and methods including optimal combinations of radial confined light energy means and mechanical effects that are added to a dilatation balloon catheter in order to improve endoluminal therapy effects, and particularly in order to facilitate the dilatation of the stenotic segment at significantly lower pressures than regular dilatation balloon catheters, without inducing collateral thermal damage to the vessel wall.

A more complete understanding of the microscopic opto-mechanical/thermo-mechanical blades attached to a dilatation balloon catheter and of the drug activation mechanism disclosed by the current invention and of the clinical advantages offered by the disclosed apparatus and methods will be readily available to those skilled in the art by reviewing the following detailed description and preferred embodiments.

### BRIEF DESCRIPTION OF THE DRAWINGS

FIG. 1 pictorially illustrates a first preferred form of a dilatation balloon catheter with externally attached optical fibers capable of emitting radial energy according to an embodiment of the present invention.

FIG. 2a pictorially illustrates the maneuvering and positioning of a dilatation balloon catheter with externally attached optical fibers capable of emitting radial energy for a PTA procedure according to the first embodiment of the present invention.

FIG. 2b pictorially illustrates the usage of a dilatation balloon catheter with externally attached optical fibers capable of emitting radial energy for a PTA procedure according to the first embodiment of the present invention.

FIG. 3a illustrates the potential energy pattern of an evanescent light-wave emitted from an optical fiber into the adjacent plaque.

FIG. 3b illustrates a first possible design of an optical fiber with a segment capable of emitting radial light-energy with high efficiency and with a substantially uniform longitudinal emission pattern.

FIG. 3c illustrates the theoretical emission pattern of the radial light emitting segment illustrated in FIG. 7b.

FIG. 4a pictorially illustrates a cross-section of a blood vessel lumen while using a dilatation balloon catheter with externally attached optical fibers capable of emitting radial energy according to the first preferred embodiment.

FIG. 4b pictorially illustrates a cross-section of a blood vessel lumen after using and retrieving a dilatation balloon catheter with externally attached optical fibers capable of emitting radial energy.

FIG. 5a pictorially illustrates the maneuvering and positioning of a dilatation balloon catheter with, externally attached optical fibers capable of emitting radial energy during a PTA procedure in a blood vessel including an asymmetric stenotic lesion.

FIG. 5b pictorially illustrates the usage of a dilatation balloon catheter with externally attached optical fibers capable of emitting radial energy during a PTA procedure in a blood vessel including an asymmetric stenotic lesion.

FIG. 6a pictorially illustrates a cross-section of a first preferred form of a dilatation balloon catheter according to an alternative design of the first embodiment of the current invention.

FIG. 6b pictorially illustrates a cross-section of a second preferred form of a dilatation balloon catheter according to an alternative design of the first embodiment of the current invention.

FIG. 6c pictorially illustrates a cross-section of a third preferred form of a dilatation balloon catheter according to an alternative design of the first embodiment of the current invention.

FIG. 6d pictorially illustrates cross-section of a fourth preferred form of a dilatation balloon catheter according to an alternative design of the first embodiment of the current invention.

FIG. 7a pictorially illustrates the light energy delivery sub-system from light source to a set of optical fibers externally attached to a dilatation balloon catheter according to the first embodiment of the present invention.

FIG. 7b pictorially illustrates an alternative design of the light energy delivery sub-system from light source to a set of optical fibers externally attached to a dilatation balloon catheter according to an alternative design of the first embodiment of the present invention.

FIG. 8 illustrates the addition of a possible bonding element to be used in conjunction with a dilatation balloon catheter with externally attached optical fibers capable of emitting radial energy.

FIG. 9 illustrates a light source to be used in conjunction with a dilatation balloon catheter with externally attached optical fibers capable of emitting radial energy.

FIG. 10a pictorially illustrates an alternative design for routing the light energy conducting elements including the externally attached optical fibers capable of emitting radial energy according to an alternative design of the first embodiment of the present invention.

FIG. 10b pictorially illustrates an alternative design for mounting a set of externally attached, optical fibers capable of emitting radial energy onto a dilatation balloon catheter according to an embodiment of the present invention.

FIG. 10c pictorially illustrates a stent delivered by a dilatation balloon catheter with externally attached optical fibers capable of emitting radial energy according to an embodiment of the present invention.

FIG. 11 pictorially illustrates a dilatation balloon catheter with externally attached optical fibers capable of emitting radial energy, and including also optical fibers capable of forward emitting light-energy according to an embodiment of the present invention.

FIG. 12 pictorially illustrates a dilatation balloon catheter with externally attached optical fibers capable of emitting radial energy according to a second embodiment of the present invention.

FIG. 13a pictorially illustrates a cross-section of a dilatation balloon catheter with externally attached optical fibers capable of emitting radial energy according to the second preferred embodiment of the present invention.

FIG. 13b pictorially illustrates a cross-section of an alternative design for a dilatation balloon catheter with externally attached optical fibers capable of emitting radial energy according to the second preferred embodiment of the present invention.

FIG. 14a pictorially illustrates the maneuvering and positioning of a dilatation balloon catheter with externally attached optical fibers emitting radial energy capable of drug activation according to the second embodiment of the present invention.

FIG. 14b pictorially illustrates the usage of a dilatation balloon catheter with externally attached optical fibers emitting radial energy capable of drug activation according to the second embodiment of the present invention.

FIG. 15 pictorially illustrates a dilatation balloon catheter with externally attached optical fibers emitting radial energy to assist the dilatation of a stenotic lesion and internal optical fibers emitting radial energy capable of drug activation according to a variation of the first embodiment of the present invention.

FIG. 16 pictorially illustrates a dilatation balloon catheter with externally attached optical means and internal optical means emitting radial energy through the external optical means according to a third embodiment of the present invention.

FIG. 17a pictorially illustrates a cross-section of an alternative design of a dilatation balloon catheter with externally attached optical means and internal optical means capable of emitting radial energy according to the third preferred embodiment of the present invention.

FIG. 17b pictorially illustrates a cross-section of an inflated dilatation balloon catheter with externally attached optical means and internal optical means capable of emitting radial energy through the external optical means according to the third preferred embodiment of the present invention.

### DETAILED DESCRIPTION OF THE DRAWINGS

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well known features may be omitted or simplified in order not to obscure the invention.

Throughout the document light means an electromagnetic wave of wavelengths is in the range of 0.2 micron to 12 micron. Throughout the document light delivering elements refer to optical fibers, hollow glass waveguides, photonic crystal fibers or other light-conductive means that may be employed to deliver light to a required position in the body in relation to the present invention. Throughout the document a light source refers to any possible means of generating light energy, such as solid-state lasers, diode lasers, gas-lasers, fiber-laser, etc.

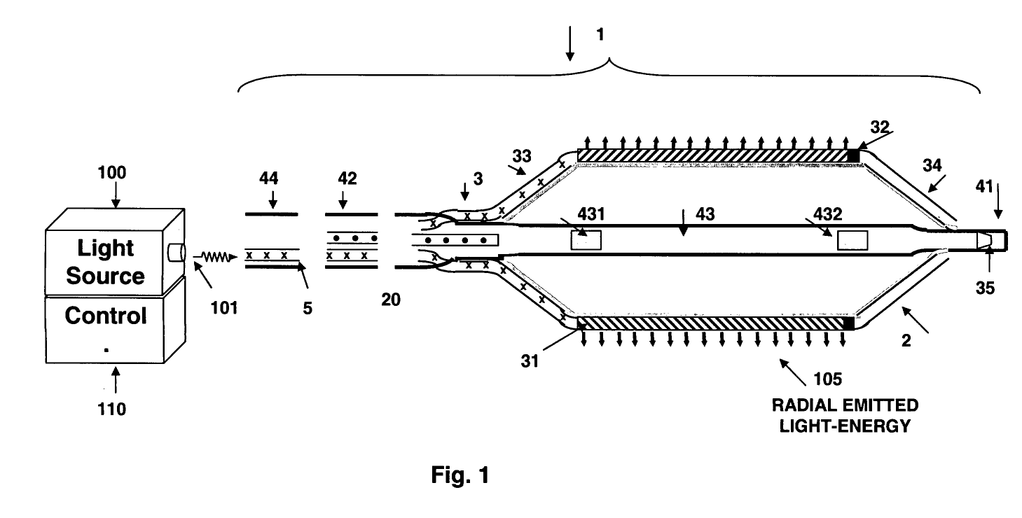

Reference is now made to FIG. 1 that illustrates a first embodiment of the present invention. A, dilatation balloon catheter 1 to be used in vascular interventions includes a basic balloon body 2 with a set of externally attached optical fibers 3. Each of the externally attached optical fibers 3 has a distal light-energy emitting section 31 capable of emitting radial light energy 105. Preferably, light-energy emitting section 31 has a diameter of 50 to 80 microns, but other diameter sizes can be used according to the vessel lumen size and clinical application. Special mechanical designs to be detailed in following sections ensure that light-energy emitting section 31 is positioned at the same location on balloon body 2, regardless of the inflation/deflation of the balloon and also ensure proper deployment and re-wrapping of set of externally attached optical fibers 3 during the inflation/deflation of balloon body 2. Alternately, light-energy emitting section 31 is longer than the working length of balloon body 2 in order to ensure that it is covering the entire balloon working length regardless of the inflation level of balloon body 2. Based on optical designs and selection of wavelength, waveform and intensity to be further detailed, radial emitted light-energy 105 has an energy pattern substantially uniform along light-energy emitting section 31 and it has a very confined interaction depth with the adjacent tissue.

An inflation system (not illustrated) enables the inflation and deflation of balloon body 2 by standard means such as supply or removal of liquid or gas. Balloon body 2 is attached to a tube-like catheter body 4 that ends in a distal catheter member 41. Tube-like catheter body 4 also includes a catheter body intermediary section 42, a catheter body intra-balloon section 43, and a catheter body proximal section 44.

Each of externally attached optical fibers 3 is optically connected to a light source 100. The connection is made either by directly connecting each fiber to the light source 100, or by means of a light-energy guide system 5.

As further detailed, the opto-mechanical design of dilatation balloon catheter 1 can include the usage of a special bonding material between set of externally attached optical fiber 3 and balloon body 2 (along its external perimeter), or the addition of elements for supporting light energy emitting section 31 on balloon body 2. Radio-opaque markers 431, 432 enable to visualize by standard fluoroscopy imaging (or other imaging modalities) the position of light-energy emitting section 31 inside the blood vessel lumen. Optional additional radio-opaque markers may be used to delimit the position of the distal catheter member 41.

Preferably, light-energy emitting section 31 ends in a totally reflecting element 32 to ensure maxim radial emission. Preferably still, light-energy emitting section 31 is coated on the side adjacent to balloon body 2 in order to decrease the total emitting section and therefore increasing the intensity of the radial light-energy emission towards the adjacent tissue. Each of externally attached optical fibers 3 also includes an optical fiber proximal section 33 that has a coating layer ensuring that no light energy is delivered by this proximal section to the adjacent anatomy.

The materials from which externally attached optical fibers 3 and optional light-energy guide system 5 are manufactured and their optical design are selected according to the selected light-source and wavelength.

Preferably, each of the externally attached optical fibers 3 terminates in an extension member 34 that continues after totally reflecting element 32. Extension member 34 is not required to transmit light energy, and can be manufactured from any suitable material, preferably an elastic polymer or plastic material, and is optionally used to improve the maneuverability of dilatation balloon catheter 1. Optionally, extension members 34 may be attached together in a distal connecting member 35. Optionally still, distal connecting member 35 can have the structure and properties of a regular guide-wire tip.

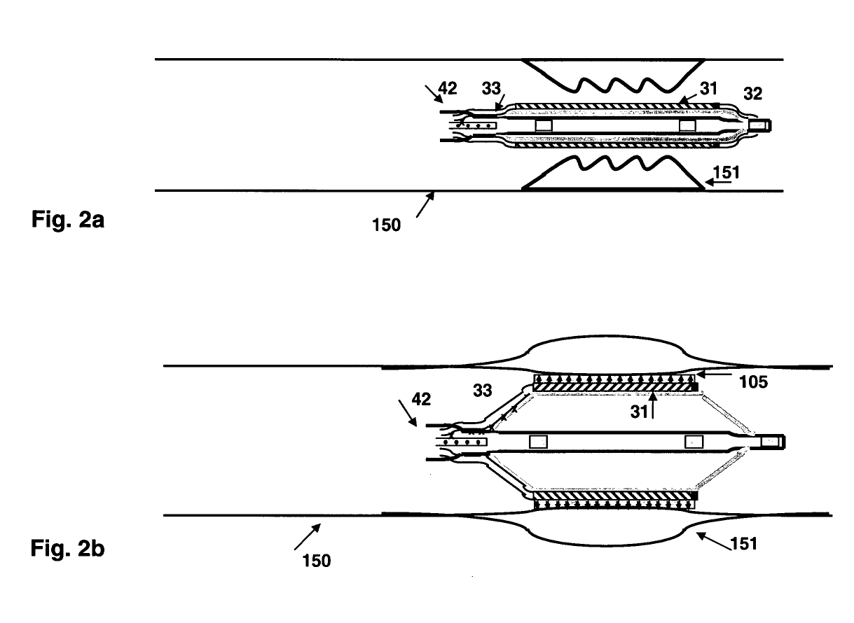

Reference is now made to FIGS. 2a, 2b that illustrate the steps in using dilatation balloon catheter 1 for achieving an optimal therapeutic effect. As a first step, dilatation balloon catheter 1 is maneuvered to position the balloon body 2 in the area of interest, i.e., the stenosed vessel including the atherosclerotic lesion 151. During the endoluminal maneuvering of dilatation balloon catheter 1 through the blood vessel lumen, balloon body 2 is totally deflated and the externally attached optical fibers 3 are fully deployed (as illustrated in FIG. 2a). This creates a flexible yet firm structure that can easily be passed through obstructing areas, such as areas where the blood vessel lumen is partially occluded by severe atherosclerotic disease. The flexible nature of the light energy-emitting section 31 enables to manufacture virtually any commonly required length overcoming length limitations that may be associated to usage of externally attached sharp-metal objects. Particularly, light energy-emitting section 31 can have a length of up to 10 cm.

Once balloon body 2 is in position, the second step is the activation of light source 100 in order to achieve an optimal energy level. Light energy is delivered through light-source optical output member 101 (and through optionally light-energy guide system 5 if used) to set of externally attached optical fibers 3. Once light energy emitting section 31 emits light energy at a required intensity level, the third step is to gradually inflate balloon body 2 by using a standard high-pressure balloon inflation device with pressure control. The gradual inflation of balloon body 2 brings light-energy emitting section 31 in contact with blood vessel wall 150 that may be covered by atherosclerotic plaque 151. Alternately, steps two and three above are interchanged, and light energy is delivered from light-source 100 to externally attached optical fibers 3 only after balloon body 2 is inflated to bring light energy emitting section 31 in contact to plaque 151 or vessel wall 150.

Further on, it might be advantageous in certain clinical conditions to activate the emission through light energy emitting sections 31 only at the beginning of the inflation process until the dilatation process successfully starts. According to such a method, once initial dilatation of the stenotic lesion is achieved the reminder of the dilatation process can be performed without emitting any energy through light energy emitting sections 31. All the embodiments described in the current invention enable switching on and off the emission from all/or part of the light energy emitting sections 31 at any stage during or after the inflation process.

The design of light-energy emitting section 31, combined with a proper selection of the light source parameters (wavelength, intensity and waveform) ensures that radial confined light energy 105 has only a very short interaction depth with the adjacent tissue. The interaction depth is defined as the depth where the energy delivered to the tissue creates one of the following effects: selected tissue removal; tissue vaporization; tissue heating and bond-weakening effect. For example, according to a preferred embodiment, for specific vascular applications the selected parameters of light source 100 and the design of light-energy emitting section 31 can ensure (as further detailed) that the interaction depth of radial confined light-energy 105 in the tissue is less than 20-50 micrometers, and that tissue at a distance of more than 50 micrometers from the light-energy emitting section 31 is not damaged. i.e. no irreversible damage is induced on tissue cells. During the inflation of balloon body 2, the externally attached light-energy emitting section 31 pressures the tissue in the exact area that was, first ablated/evaporated/bond-weakened by the effect induced by the radial light energy 105. Therefore, during the inflation process, externally attached light-energy emitting section 31 facilitates the splitting of the atherosclerotic plaque or thrombosis through a combined effect of confined radial energy delivery and complementary mechanical effects that together cause longitudinal microscopic “cuts” or “cracks” in the plaque.

The dilatation balloon catheter 1 relying on the microscopic opto-mechanical effect described above (and further disclosed in the detailed description) can easily dilate rigid, elastic or diffusely diseased segments, and ensures a smooth dilatation process at lower pressure if matched by a proper selection of the balloon material and design and by the inflation rate. Dilatation balloon catheter 1 can be used as a stand-alone treatment or in conjunction with other treatment options such as stenting.

Additional optional therapy effects can be achieved by emitting radial light-energy after the successful inflation of balloon body 2 in order to create longitudinal microscopic thermal effects that can prevent generation of prolific hyperplasia and therefore acute, restenosis following the PTA procedure. For example it is possible to change the intensity and waveform and/or wavelength of the delivered light energy in order to weld the microscopic induced cuts.

Reducing the required dilatation pressure during the inflation process and reducing the induced barotrauma to the vessel wall is of high importance in reducing restenosis effects. Proper matching of the material and the design of balloon body 2 in combination with a matched inflation rate enable to benefit from the microscopic opto-mechanical effects induced in the stenotic tissue by light-energy emitting section 31, enables to dilate stenotic lesions at significantly lower pressures than regular dilatation balloons, and particularly should enable dilating stenotic lesions at pressure levels between two to five, atmospheres.

Preferably, the material from which balloon body 2 is manufactured ensures that balloon body 2 acts as a semi-compliant balloon at lower pressure levels and acts as a low-compliant or non-compliant balloon at higher pressure levels. Particularly, such a design ensures that at lower pressures the balloon body 2 is capable of expanding and, dilating the plaque assisted by the opto-mechanical effects induced in the plaque by light-energy emitting section 31, while having a strong texture capable of supporting the mechanical pressure exerted by light-energy emitting section 31, and avoiding the risk of over-stretching or injuring vessel wall 150 at higher pressures. Alternately, balloon body 2 is manufactured from low-compliant or non-compliant materials or from a mix of layers that ensures low-compliance or non-compliance properties. Optionally still, balloon body 2 is manufactured from semi-compliant materials, or from a mix of layers that ensures semi-compliant properties.

According to a preferred method of operation, the inflation rate takes into account the size and the material of balloon body 2, and the pressure in balloon body 2 is not increased as long as the inflation rate matches the expected rate of “cracking or scoring” created by the opto-mechanical effects induced in the stenotic plaque by light-energy emitting section 31. There are various methods for adjusting the dilatation rate to the opto-mechanical effect, and some of them are further described. According to a first preferred method and apparatus the control unit 110 automatically measures and correlates between the pressure in balloon body 2, and its size (diameter, etc) as a function of time. According to an alternative semi-automatic method, control unit 110 indicates to the user the inflation rate vs. the expected rate and the user decides whether to increase the pressure in the balloon 2. According to still further alternative methods the user knows the expected inflation rate as a specification of the system, and gradually increases the pressure in the balloon 2 according to the expected rate. Additionally, the value of the expected inflation rate at a certain pressure might be dependent on the type of lesion and additional parameters that are automatically detected or manually indicated (such as type of vessel, level of calcification, etc).

The pressure in balloon 2 is induced and measured by standard methods (all the inflation systems currently commonly used to inflate dilatation balloon catheters accurately indicate the pressure in the balloon) or by various alternative methods readily available for the skilled in the art. The shape/diameter of balloon body 2 can be measured by various methods and apparatus as further described. According to a first preferred method, the shape/diameter of the balloon body 2 is measured by imaging modalities, such; as extraluminal imaging modalities: X-Ray images produced by angio systems or MR-angio images during the inflation of balloon 2 or by endoluminal imaging modalities (IVUS, optical imaging, thermal imaging, intraluminal MR imaging, etc). According to alternative method the diameter of balloon body 2 is measured by elasto-mechanical means. According to still alternative methods, the shape of balloon 2 is measured by using the optical fibers themselves or additional optical fibers for example by methods used by the Measurand company to measure shape.

According to alternative methods the dilatation rate is updated based on time considerations. According to a first preferred method and apparatus, control unit 110 automatically increases the pressure in balloon body 2 to a certain level based on time considerations that are made according to the expected effect of light-energy emitting section 31. According to an alternative method, control unit 110 indicates to the user based on time measurement, that it should increase the pressure by a certain amount. According to still further options, the user gradually increases the pressure in the balloon according to known values provided in accordance to the performance of the system. The required time intervals for increasing the pressure in the balloon might be dependent on the type of lesion and additional parameters that are automatically or manually indicated (such as type of vessel, level of calcification, etc).

According to a still alternative method, the user can start and stop the delivery of light-energy through light-energy emitting section 31 according to the conditions encountered during the intervention. Moreover, control unit 110 can automatically cease the emission form all/part of the light delivery elements when the inflation level/pressure has received a certain level or when balloon body 2 has reached a certain size in order to prevent unwanted damage to the vessel wall 150.

Optionally, the light energy delivery process can be repeated several times during the inflation in order to minimize risks, and in order to increase the efficiency particularly in cases of severe atheromatosis or heavily calcified atherosclerotic plaque. In severe cases, if required, balloon body 2 can be re-inflated, and the delivery of radial confined light energy: can be repeated to a required vessel segment. Still optionally and if clinically required, balloon body 2 can be rotated in order to create multiple longitudinal opto-mechanical microscopic incisions or tissue “cracks”. Still optionally, different waveforms and energy levels can be employed at different stages of the inflation-process in order to achieve an optimal clinical result. Still optionally, in cases where well organized plaque or heavy calcification obstructs the inflation of balloon body 2, delivery of light energy can be performed in different forms (wavelength/level of energy/duration of pulse) during the inflation in order to facilitate the recanalization process, and to minimize the risk of complications.

As mentioned in previous sections, the parameters of the emitted light energy wavelength, waveform and intensity, together with the design of the light-energy emitting-section 31 define the type of light-tissue interaction and the interaction depth. According to a first preferred option, the energy emitted by light-energy emitting section 31 is in the form of pulses with high fluence levels ensuring that most or all of the absorbed energy is converted into tissue vaporization and ejection of products. This operation mode will be further referenced as Collateral-Damage-Free Ablation (CDFA), and is a preferred mode of reducing restenosis due to lack of thermal damage to the tissue of the vessel wall. CDFA is preferably achieved with light pulses shorter than thermal diffusion period, where each pulse achieves the required effect, and with a repetition interval between the pulses that is longer than the thermal diffusion period. A preferred alternative waveform includes the emission of packages of very short energetic pulses (each package of pulses includes several very short pulses), such that the duration of each package of pulses is shorter than the thermal diffusion period and the interval between two packages of pulses is larger than the thermal diffusion period. The effect of each package of pulses causes CDFA. Such a package of pulses can be achieved by using one source or several sources emitting in parallel through the same output and being synchronized to sequentially emit pulses, such that the delay between the sequential emissions of different sources satisfies the above conditions.

Preferably still, CDFA is performed at wavelengths where the absorption coefficient of plaque is high (the penetration depth of the light energy is small), and ensures that only a very controlled and confined volume of tissue is affected. For example, using wavelengths in the UV band (below 0.32 microns) or in the IR (above 2.2 microns) ensures a small penetration depth per pulse (magnitudes of microns up to several tens of microns) and reduces the amount of energy required per pulse. For example, using an Er:Yag source at 2.9 micron would require light-energy emitting section 31 to emit a fluence level in the range of 0.7 J/cm² to 3.0 J/cm² (or above) per pulse. The selection of the wavelength can be based on choosing wavelengths that are strongly absorbed by most types of encountered tissue, such as UV (0.308 micron, for example) or IR-(2.2 micron and above) or on choosing wavelengths that are highly absorbed by specific cromophores existing in the plaque or tissue to be dilated. For example, in tissue, with a high enough content of blood it is possible to use light wavelengths that are highly absorbed by hemoglobin (for example, wavelengths between 0.4 to 0.6 micron).

Optionally, in order to effectively use wavelengths that are sub-optimal in regular usage—i.e.,—have a large penetration depth in tissue (small absorption coefficient)—an alternative method is to use a substance that when, for example, impregnated in the balloon body 2 and released during the light interaction process increases the absorption coefficient of the tissue for the specific wavelength and decreases collateral damage. An optional substance is Hematoporphyrin (HPD) that is documented for increasing the absorption of plaque for specific light wavelengths. Alternative methods may even include oral Carotene that seems to be selectively retained in plaque or other substances being delivered prior or during the procedure through the balloon catheter or other percutaneous or no-percutaneous methods.

CDFA effects can also be achieved when light source 100 is a laser source capable of emitting very short laser pulses with high-peak power. In this case, the CDFA effect is achieved by thermo-elastic effects and/or ablative recoil (also known as photoacoustic effect), or is achieved by multiphoton absorption that leads to ionization and optical breakdown of the tissue. A private case involves the usage of a femtosecond laser capable of emitting high, peak-power, femtosecond laser pulses.

According to a second preferred option, the radial energy emitted by light-energy emitting section 31 is designed to cause a confined volume of adjacent tissue to reach a temperature of 60° C.-80° C. (and in some cases slightly above 80° C.). In order to achieve this thermal effect without inducing thermal damage at larger depths, the optimal way is to select wavelengths with small penetration depth in the relevant tissue (plaque and vessel wall) and to use a pulsed waveform, such that each pulse has enough intensity to achieve a required thermal effect and the repetition interval between pulses is longer than the thermal diffusion period. This selection of wavelength, waveform and intensity ensures that no thermal damage is induced on further located tissue, due to thermal diffusion in the tissue. For example, using a pulsed Er:Yag source at 2.9 micron would require light-energy emitting section 31 to emit a fluence level of around 20-35 mJ/cm² per pulse, and would heat a tissue volume with a depth of less than several microns. Heating a small longitudinal volume of tissue combined with the pressure exerted by light-energy emitting section 31 on the same exact tissue volume facilitates creation of cracks or causes bond-weakening of the tissue; and therefore facilitate the dilatation of balloon body 2. Other various wavelengths, preferably in the range below 0.55 microns or above 1.5 microns are suitable for such heating of tissue. An alternative preferred waveform includes the emission of packages of very short pulses (each package of pulses includes several very short pulses), such that the duration of each package of pulses is shorter than the thermal diffusion period and the interval between two packages of pulses is larger than the thermal diffusion period. The thermal effect of the package of pulses would be equivalent, to the thermal effect of a larger duration pulse. Such a package of pulses can be achieved by using one source or several sources emitting in parallel through the same output and being synchronized to sequentially emit pulses, such that the delay between the sequential emission of different sources satisfies the above conditions. Alternately still, under specific clinical conditions, the energy emitted by light-energy emitting section 31 for heating a radial confined volume of tissue may be in the form of longer light pulses or even continuous emission for short periods of time (up to several seconds) where the only constraint is ensuring that no thermal damage is induced to the tissue of the vessel wall. This might be implemented, for example in cases where at the beginning of inflation process light-energy emitting section 31 is at least several hundred of microns from blood vessel wall 150.

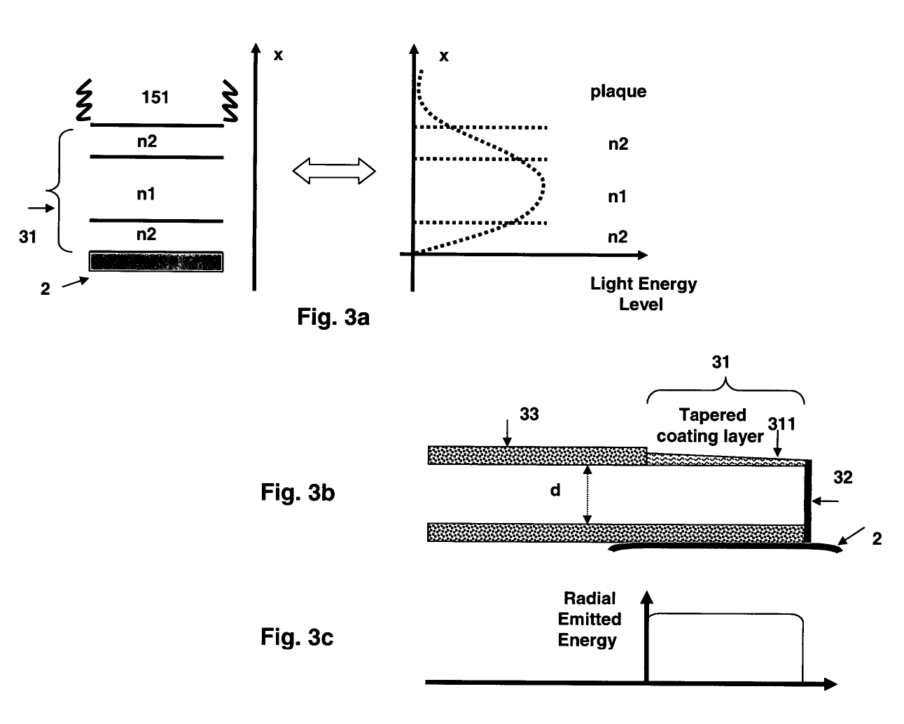

As described in the above sections, it is possible to select and use wavelength, waveform and intensity levels such that the radial light energy 105 has a very confined interaction depth and does not induce spread thermal damage to the vessel wall. In order to avoid thermal damage and ensure the efficacy of the device it is also important to ensure that the intensity of the emitted radial light energy is longitudinal and angular uniform (or quasi-uniform) along light-energy emitting section 31. FIG. 3a is illustrating the fading energy profile of an evanescent light wave. As illustrated in FIG. 3a, if the delivery of light-energy to the tissue is made in the form of an evanescent wave, the significant energy delivery in tissue is confined to a very small volume adjacent to light-energy emitting section 31, and this volume of adjacent tissue can be as small as magnitudes of several microns or less. The main problem with the usage of evanescent wave methods is that the optical coupling efficiency in tissue is low, and that the increase in the optical coupling is highly dependent on having a proper angle between the light propagation direction and the interface between the optical fiber and the tissue. Moreover, slight changes in this angle can result in a poor uniformity along the length of light-energy emitting section 31 thus leading to lack of repeatability and even to unwanted tissue thermal damage. Having a low tissue coupling efficiency also increases the light-energy level required for achieving the desired, therapeutic effect, i.e. requires a larger light source, larger light-delivery elements with a larger-damage threshold and causes unwanted heat and ultimately thermal collateral damage that could eventually lead to increased levels of restenosis.

There are several radial light-energy delivery methods and optical designs of light-energy emitting section 31 capable of emitting radial light-energy with a high efficiency rate and capable of achieving the required energy confinement without inducing collateral damage effects. These methods and designs ensure uniform light-energy delivery along the radial light-energy emitting section 31, which is a critical feature in designing a robust and repeatable apparatus.

According to a first preferred method and apparatus, illustrated in FIG. 3b, the cladding of radial light-energy emitting section 31 is removed on the side facing the tissue, and a thin layer of higher refractive index material 311, for example optical epoxy is used in order to achieve a uniform scattering effect over the entire radial light-energy emitting section 31. The thin layer of higher refractive index should preferably be made from a mixture of at least two materials with different refractive indexes (one significantly different from the other) in order to couple out the energy outside the optical fiber. In order to achieve uniform amount of energy delivery along radial light-energy emitting section 31, the output-coupling factor should increase along its length. This can be achieved in the case of using the thin coating layer of higher refractive index by changing the thickness of the coating layer 311 or the mixture percentage along the length of the fiber. Preferably, the cladding on the side attached to the balloon body 2 is left intact in order to ensure maximal reflectance towards the tissue. Optionally, a thin metal layer ensuring an even higher reflectance coats the fibers side attached to the balloon body 2. The design illustrated in FIG. 3b ensures a high level of longitudinal and angular uniformity of the emitted light energy—as illustrated in FIG. 3c—that is imperative for achieving repeatable results.

Alternative designs and apparatus for radial light-energy emitting section 31, include design of a special dispersive fiber core, for example by usage of specially designed hollow light-guides or all-dielectric fibers consisting of a periodic array of air holes in silica (similar in concept although opposite in result to designs of photonic crystal fibers). Still alternative optical designs (not illustrated) for emitting a radial confined light-energy are based on the usage of an optical fiber with a tapered thinner cross-section (core and/or cladding) for the radial light-energy emitting section 31 causing dispersion of a light wave in this thinner section. According to still alternative techniques radial-light-energy emitting section 31 has preferably a roughened surface on the side facing/in contact to the tissue. According to still alternative designs—not illustrated—radial light-energy emitting section 31 consists of a group of internal reflecting (or partially reflecting) surfaces that direct laterally the light energy towards the tissue. If implemented group of internal reflecting surfaces would have increasing size towards the distal part of the radial emitting section 31 in order to ensure longitudinal uniform illumination. According to still alternative designs—not illustrated—radial light-energy emitting section 31 consists of a group of internal optical refracting surfaces or elements that direct laterally the light energy towards the tissue. If implemented the optical refracting surfaces or elements would ensure the longitudinal uniformity required.

It is particularly advantageous to limit the total area through which the radial emission occurs in order to reduce the collateral damage and in order to reduce the required amount of energy required from the laser source. Given that the required fluence for achieving a required effect is Fluence Threshold (J/cm²), the total required energy is:
Total_{—Energy}=Fluence_{—Threshold}(J/cm²)*Length_{—Arc}(cm)*Length(cm)
where Length(cm) is the length of light energy-emitting section 31 and
Length_{—Arc}(cm) is the length of the arc used for radial emission.

In order to reduce the required energy levels it is possible to decrease the arc through which the radial emission is performed. For example, when using optical fibers with a diameter of 80 microns it is possible to limit the radial emitting section to an arc of less than 25 mincrons, design that requires only ⅕ of the energy required to emit through the entire, 180 of the upper arc of light-energy emitting section 31. Optionally, (not-illustrated) radial light-energy emitting section 31 consists of longitudinally interlaced sections that emit radial light-energy and sections that do not emit radial light-energy. Given that the sections not emitting are small enough the mix is capable of achieving the required effect while reducing the required total energy. Such a design can be achieved for example, by using a group of internal reflecting (or partially reflecting) surfaces or a group of optical refracting surfaces or elements as described in the previous paragraph.

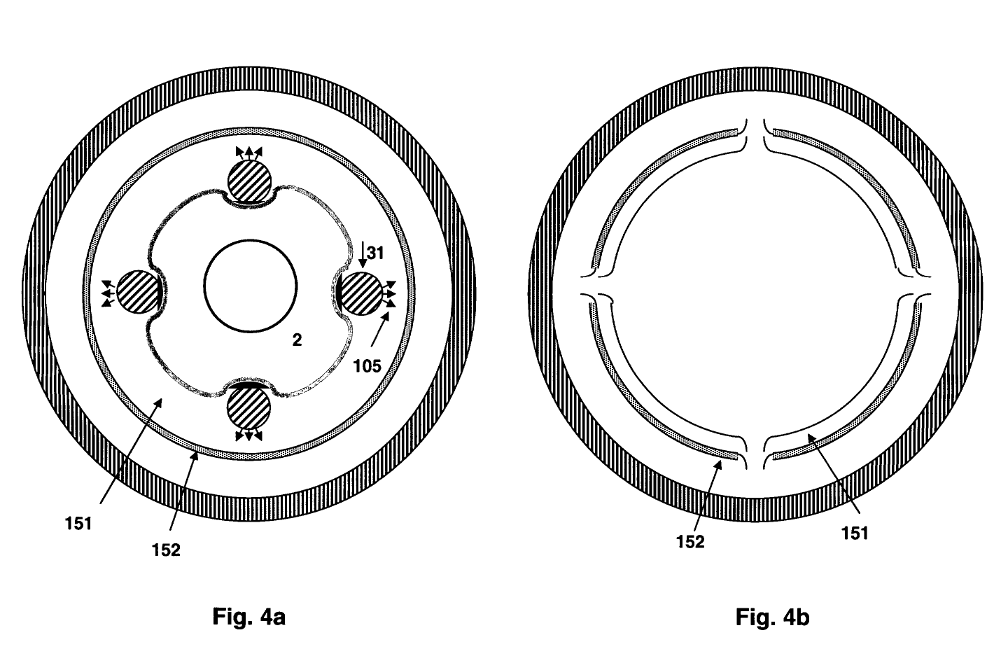

The clinical effect on blood vessel wall 150 and atherosclerotic plaque 151 is illustrated in FIGS. 4a and 4b. The illustrated cross-section of blood vessel wall 150 clearly shows the expected cuts of atherosclerotic plaque 151 and intima layer 152 that are the result of the combined mechanical and radial light energy delivery effect of externally attached optical fibers 3.

Dilatation balloon catheter 1 can be used regardless of whether a stent has already been placed in the area to be treated without danger of mechanical obstruction or other interference. Particularly, dilatation balloon catheter 1 with externally attached optical fibers 3 ensures the same clinical benefits when used for treating in-stent restenosis, i.e. dilating stenosis that occur in a stented vessel segment.

To summarize, a dilatation balloon catheter 1 with externally attached optical fibers 3 as described in the previous: embodiments, based on the selection of the light parameters, balloon materials and clinical workflow and method described in the previous sections provides several important clinical advantages:
It overcomes the dilatation of rigid, elastic or diffusely diseased segments;
It is equally suitable for passing tortuous vessels, treating ostial lesions and in-stent restenosis;
It facilitates dilatation of stenotic lesions at low pressure levels by scoring or inducing cracks or bond-weaken segments in the lesion that assist the dilatation process.

It reduces the trauma induced to the vessel wall during the inflation process and it causes discontinuities in the lesion and therefore, it lowers the chances for hyperplasia, it prevents abrupt closure of the dilated segment and it significantly lowers the incidence of restenosis after successful expansion of the blood vessel lumen.

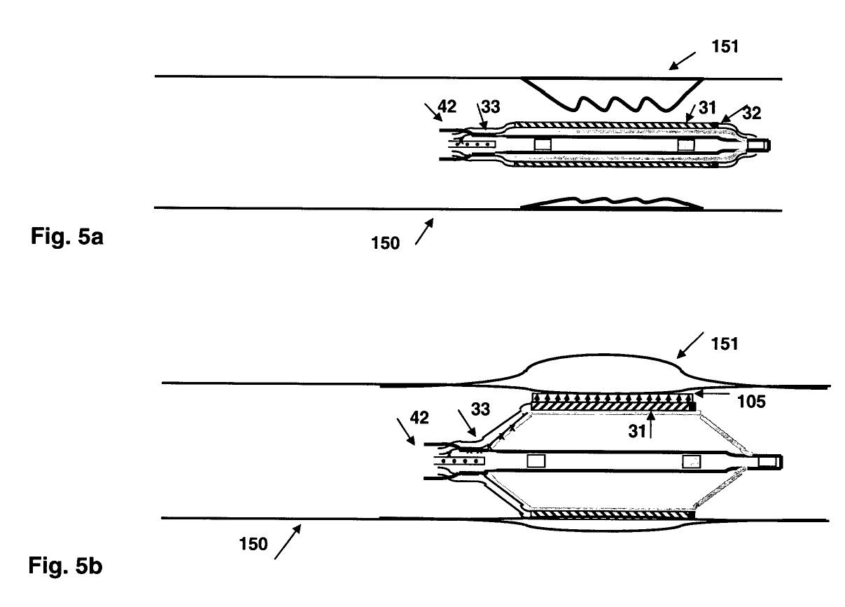

Reference is now made to FIG. 5a and FIG. 5b that illustrate a method for the activation of a dilatation balloon catheter 1 with externally attached optical means. Blood vessel lumen presents an asymmetric/eccentric stenotic region as illustrated. In this case, it is optimal to emit light-energy only through light energy emitting sections 31 that are facing the larger volume stenotic plaque 151. Such a selective activation has clinical advantages and prevents unnecessary damage to blood vessel wall 150.

The selective activation can be performed for all the embodiments described in the current invention where there is a separate/or at least partially separate channel for each/or for sub-groups of externally attached light delivery elements.

Several methods can be used in order to assess the asymmetry in the stenotic plaque. These methods include but are not limited to IVUS imaging, endoluminal imaging modalities based on MR imaging, endoluminal imaging modalities based on optical reflection or other optical effects, external imaging modalities such as MRI.

According to one preferred embodiment light energy emitting sections 31 or other optical elements are used for determining the nature of the tissue adjacent to them. This can be performed by various infrared (IR) spectroscopic methods such as attenuated total reflectance (ATR) Fourier Transform IR (FTIR). This type of IR spectroscopy which is based on measuring the absorption of totally internally reflected IR beam when the beam becomes into contact with tissue, and therefore is most adequate for determining the type of tissue adjacent to light energy emitting sections 31. It is even possible to automatically control the selective/non-selective emission based on the real-time spectroscopic measurements produced before, during and after the inflation process.

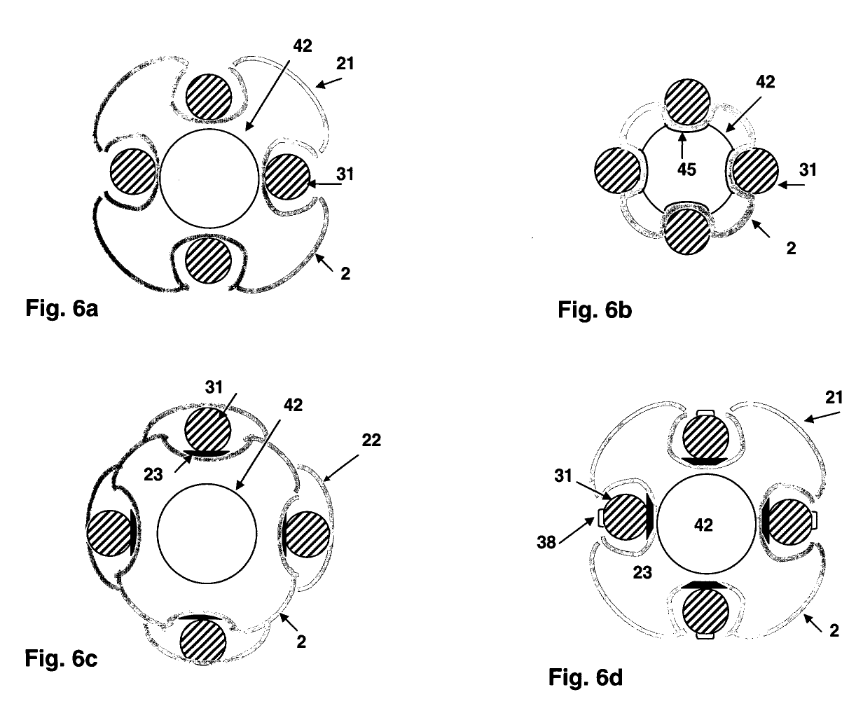

The design of the balloon body 2 and the selection of its material takes into account the existence and action of externally attached optical fibers 3. Reference is now made to FIGS. 6a-6d. Optionally, balloon body 2 can include folding wings 21 as illustrated in FIG. 6a, or can include cavity-deformation 45 in catheter body intra-balloon section 43. Folding wings 21 or cavity-deformations 45, if implemented, ensure that while balloon body 2 is not inflated it contains at least partially light energy emitting section 31. These optional modifications can improve maneuverability during the endoluminal manipulation of dilatation balloon catheter 1, preventing potential damage to blood vessel wall 150 during the transition phase. More particularly, the addition of the folding wings 21 or of the cavity-deformations 45 is a precaution mean that also facilitates the bonding of the light-energy emitting section 31 to balloon body 2. Optionally, as illustrated in FIG. 6c, balloon body 2 includes external membrane 22 over the light-energy emitting section 31. Each external membrane 22 together with balloon body 2 creates a separate chamber that contains the light-energy emitting section 31. If implemented, the separate chamber is made from a material being transparent for the radial light energy emitted by light energy emitting section 31, and at the same time having properties substantially similar to the material of balloon body 2 in terms of smoothness. Optionally, the separate chambers are part of balloon body 2, and in this case light energy emitting section 31 can be considered as being embedded in balloon body 2. External membrane 22 constitutes an optional additional precaution mean to protect healthy vessel wall tissue during the transition of dilatation balloon catheter 1 through the vessel lumen.

Reference is now made to FIG. 6c, and FIG. 6d that present additional mechanical methods for improving the opto-mechanical effect of externally attached optical fibers 3. FIG. 6c illustrates the option of using a basis 23 for supporting light energy emitting section 31 where basis 23 are attached onto or embedded in balloon body 2. Basis 23 can be made of a variety of materials, and can have various shapes and levels of elasticity ensuring that it does not obstruct the inflation/deflation process while providing additional support and strength to the balloon section below externally attached optical fibers 3. Still optional (not illustrated) light energy emitting section 31 has a special shape with a wider base in order to gain better support from the balloon body 2. FIG. 6d illustrates the option of adding/shaping the light energy emitting section 31 with a protrusions 38 matched to the angular section that emits radial light-energy. Protrusion 38 is transparent to the radial emitted light energy and optionally can assist in focusing the radiated light. Additional options (not illustrated) include usage of special shape optical designs for the light energy emitting section 31, such as elliptic optical fibers, shaped to optimize the opto-mechanical effects.

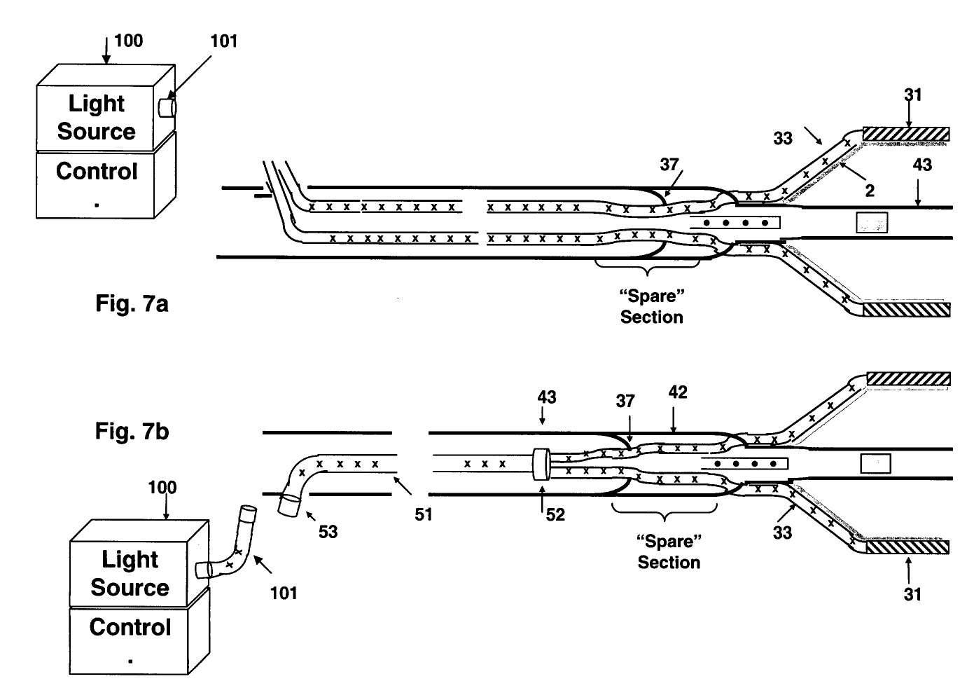

Proper operation of dilatation balloon catheter 1 requires that light-energy emitting section 31 is positioned on the non-tapered section of balloon body 2 regardless of the inflation level of balloon body 2, and that radio-opaque markers 431 and 432 indicate at all time the position of light energy emitting section 31. Reference is now made to FIG. 7a and FIG. 7b that illustrate mechanical designs ensuring the proper wrapping and re-wrapping of externally attached optical fibers 3. In FIG. 7a each externally attached optical fibers 3 is connected separately to the laser source 100, whereas in FIG. 7b each externally attached optical fibers 3 is connected to a light-energy guide system 5 that includes an intermediary optical guide member 51, a proximal optical connector 53 and a distal optical connector 52. As illustrated in FIG. 7a, and in FIG. 7b optical fiber proximal section 33 has a spare segment that enables to inflate balloon body 2 while ensuring that light-energy emitting section 31 maintains its position on balloon body 2 regardless of the inflation level of balloon body 2. The spare segment can be routed inside or outside catheter body intermediary section 42. Optionally, the spare segment can be connected through an elastic attachment member 37 such as flexible hoops attached to tube-like catheter body 4 or routed through a flexible conduit. The “spare” section of optical fiber proximal section 33 together with the optional elastic attachment member 37 or other similar mechanical elements enables to inflate/deflate the balloon body 2 while keeping externally attached optical fibers 3 attached onto balloon body 2 and then re-wrapping them properly.

According to still an alternative optical design (not-illustrated) the externally attached optical fibers 3 are branches from light-energy guide system 5 that consists of an optical fiber of larger core.

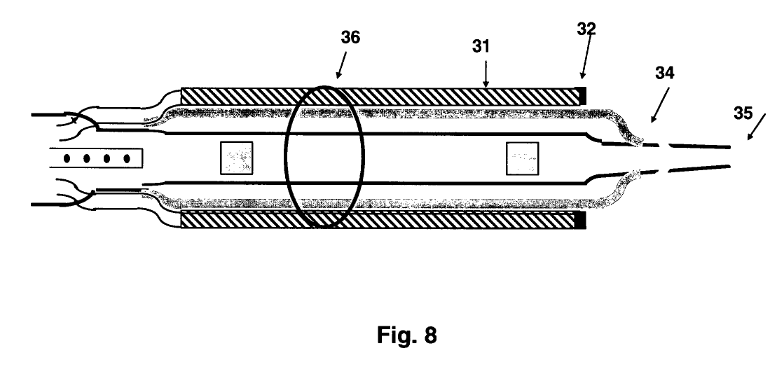

Various other mechanical designs based on having a spare segment of optical fiber proximal section 33 can be implemented in order to ensure that light-energy emitting section 3 is positioned on the non-tapered section of balloon body 2 regardless of its inflation level. For example, as illustrated in FIG. 8, one or more fiber bonding elastic elements 36 are optionally used in order to ensure that set of externally attached optical fibers 3 remains attached to balloon body 2 during the operation, and particularly that the externally attached optical fibers 3 and the balloon body 2 re-wrap properly after being deflated.

According to an optional alternative design—not illustrated—balloon body 2 has a “dog bone” shape that when inflated seals the area to be treated. The “dog bone” shape can also be achieved by using three separate balloons where the first and the third are inflated at the beginning of the procedure in order to seal the area to be dilated and the second (middle) balloon including the set of externally attached optical fibers 3 is used for a smooth dilatation of the stenotic area as described through out this document. Still, optionally, saline or other biocompatible substance that transmits the light wavelength is used to flush (constantly or periodically) light-energy emitting section 31 in order to ensure that no residual blood or tissue parts are gathering on the emitting section. Alternately, balloon body 2 can have alternative shapes that can be useful for specific clinical applications.

An additional optional modification to the dilatation balloon catheters 1 described in this invention may include the usage of a multi-lumen dilatation balloon catheter 1 where at least one lumen is used for allowing blood to profuse during the entire dilatation process.

An additional variation to the embodiments above is now described. A light energy guide system 5 includes an intermediary optical guide member 51 that is a liquid light-guide capable of transmitting light-energy to set of externally attached optical fibers 31 through distal optical connector 52. The usage of a liquid light-guide, can be implemented in several ways. According to one preferred embodiment the liquid used for inflating the balloon body 2 is also used as a light-guide. The tube-like catheter body-starts conducting light after the inflation of the balloon. In this case, the same connector can be used to inflate the balloon and to connect light-source 100 to light energy guide system. Alternately, the liquid used for inflating the balloon is introduced/taken-out through a standard lumen that is unrelated to the liquid wave-guide 5.

The endoluminal maneuvering of balloon body 2 to a desired position may be implemented in various ways. For example, the maneuvering and transport system can be a regular “MonoRail” angio mechanism including a guide-wire that is passed through a bore in the distal part of catheter body intermediary section 42, passes next to the distal part of inflation system 20 and continues through a bore in distal catheter member 41. An alternative mechanism is the “Over-the-Wire” method. In this case, the guide-wire is inserted through a guide-wire insertion connector connected to the proximal tube-like catheter lumen. An alternative design could be the usage of a “MiniRail” transport system (MiniRail is a trademark of X-Technologies Ltd.).

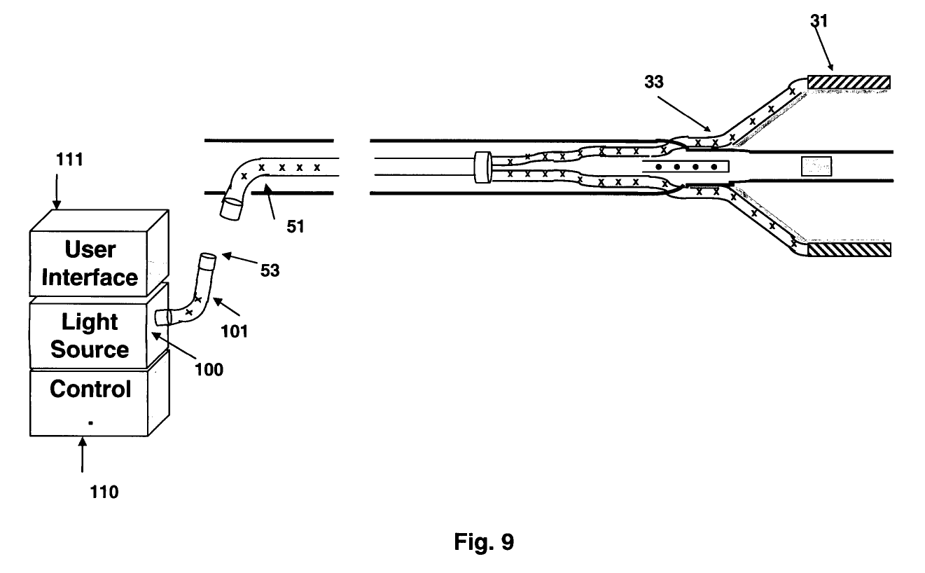

Reference is now being made to FIG. 9. Light source 100 is capable of emitting light energy at one or more specific predefined wavelengths. Light source 100 can be based on a single light source or several light-sources used in parallel or sequentially. Optionally, several wavelengths can be emitted in parallel by using multiple sources or by using an Optical Parametric Oscillator (OPO) module from a single source or beam splitting optics. As mentioned in previous paragraphs, light source 100 preferably emits light at a wavelength for which the plaque or other relevant tissue has a high absorption coefficient. Preferably still, the selected wavelength(s) has (have) a higher absorption coefficient in plaque than in the vessel wall. Still preferably, and as previously stated, light-source 100 emits pulses with duration shorter than the thermal diffusion rate (of the tissue to be treated) and with a repetition interval between the pulses that is longer than the thermal diffusion rate of the relevant tissue. Alternately, light source 100 emits a packages of very short energetic pulses (each package of pulse includes several very short pulses), such that the duration of each package of pulses is, shorter than the thermal diffusion period and the interval between two packages of pulses is larger than the thermal diffusion period. Such a waveform can be achieved by, using a single source or several synchronized sources with a common output and used sequentially. A private case of such a waveform includes usage of femtosecond lasers and femtosecond laser pulses. The therapeutic effect is achieved in this case by multiphoton absorption that leads to ionization and optical breakdown of the tissue.

Light source 100 includes a power supply unit and an optional battery that would ensure at least a limited stand-alone operation of light source 100 without the need to connect it to power during the intervention. Light source 100 is of moderate size, enabling to be placed in the working area without obstructing the user or even to be held by the user during the intervention. Light source 100 may or may not be disposable after one or more interventions.

According to still ter aspects of the current invention the operation of light-source 100 is controlled by means of a control unit 110. Control unit 110 includes a user interface, and optionally correlates (automatically or semi-automatically) the operation of light-source 100 to the inflation rate of balloon body 2. The correlation can be based on methods, sensors and algorithms as described in previous paragraphs. Control unit 110 also coordinates al the built-in-tests based on apparatus and algorithms for detecting failures in the light-source 100 or in the externally attached optical fibers 3. This is particularly important in cases where the optical fibers 3 are involuntarily damaged. In cases of failure, improper operation or damage of light-source 100 and/or of externally attached optical fibers 3, the control unit 110 issues, an alert to the user through a user interface 111 and/or automatically stops the operation of light-source 100.

In order to reduce the amount of peak power and average energy required from light-source 100 it is optionally possible to multiplex the light emission between externally attached optical fibers 3 by standard optical means. This is particularly useful when employing high-peak-power levels required for Collateral-Damage-Free Ablation (CDFA). For example, when using four externally attached optical fibers 3 it is possible by multiplexing two fibers at a time to reduce the peak power by half.

Control, unit 110 preferably recognizes automatically the length of light-energy emitting section 31 and/or size of balloon body 2, for example by including an encoded element in proximal: optical connector 53, where the code indicates the type of balloon and its working-length. For example, for a 3 cm length (of the light-energy emitting section 31) the required overall power will be twice the overall power for a 1.5 cm length. Alternately, the user can indicate the relevant parameters (type of balloon, etc) through: user interface 111, and control unit 110 adjusts accordingly the energy levels and waveforms and optionally even the wavelength. Similar automatic methods can be used if required in order to switch-on/switch-off or regulate the light intensity level according to the pressure level/inflation diameter of balloon body 2.

Optionally, control unit 110 is able to adjust waveform/intensity or even select the wavelength according to the type of tissue adjacent to the radial light-energy emitting section 31. Recognition of the type of tissue can be done by means of medical imaging devices such as X-Ray, Doppler Ultrasound, CT-Angiography, MR-Angiography, IVUS or other suitable imaging devices. Optionally, light-energy emitting section 31 or additional optical elements enable to establish the type of tissue by spectroscopic methods. Still optionally, control unit 110 and user interface 111 enable the user to manually indicate the type of vessel (for example, coronary, peripheral, etc) and type of tissue (for example, yellow plaque, calcified, etc), and control unit 110 adjusts accordingly the waveform and intensity or even selects the wavelength accordingly.

According to further variations, the entire dilatation balloon catheter 1 and light-energy guide system 5 are MR, compatible and light-source 100 can be used inside an MRI suite, for performing intraluminal interventions with the assistance of MR imaging. Particularly, dilatation balloon catheter 1 and light-energy guide system 5 can be used inside an open or closed MR system. Radio-opaque markers 431 and 432 would be replaced in such an application by MRI visible markers. For, such an intervention, specific MR protocols can be utilized for monitoring the temperature around the “light-energy” blades.

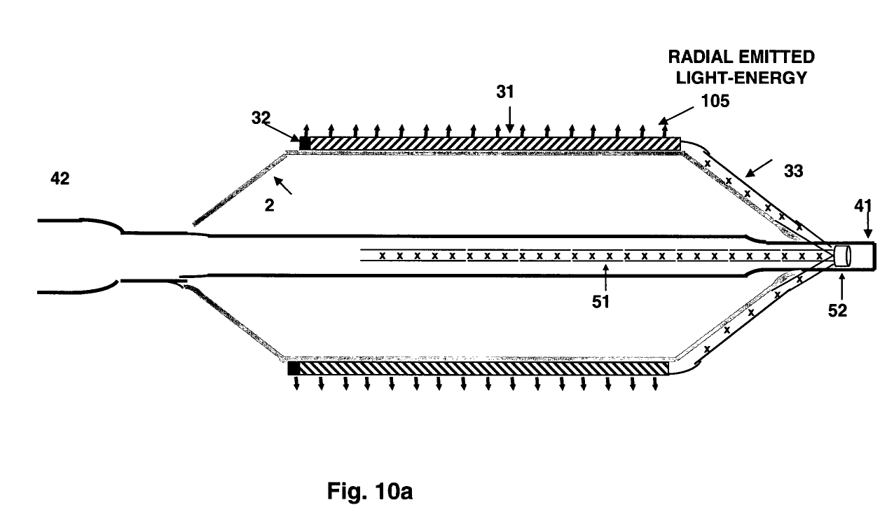

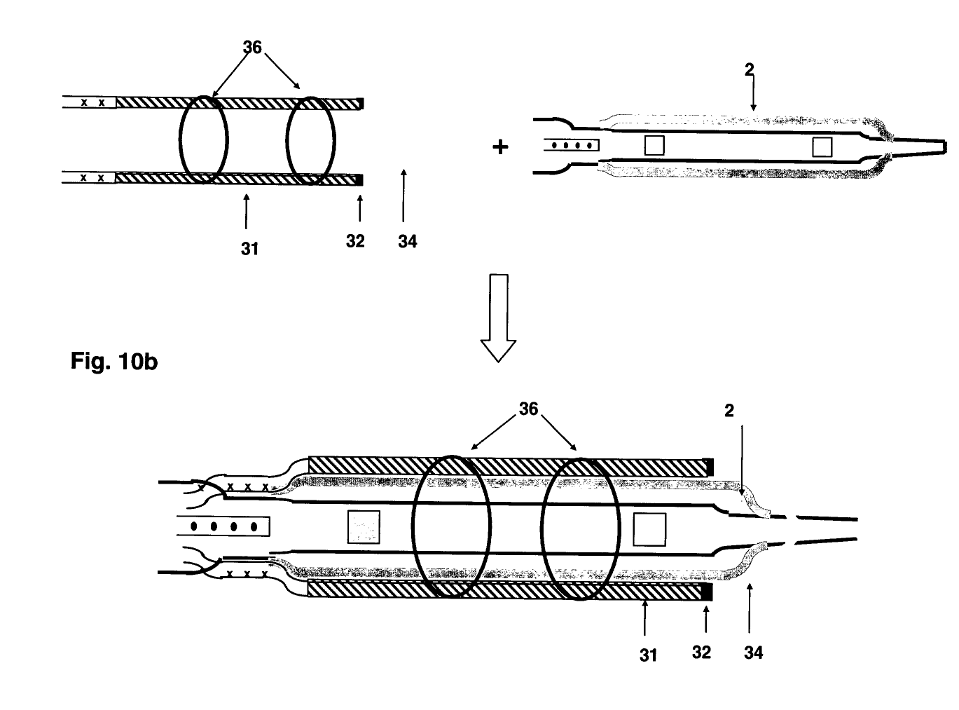

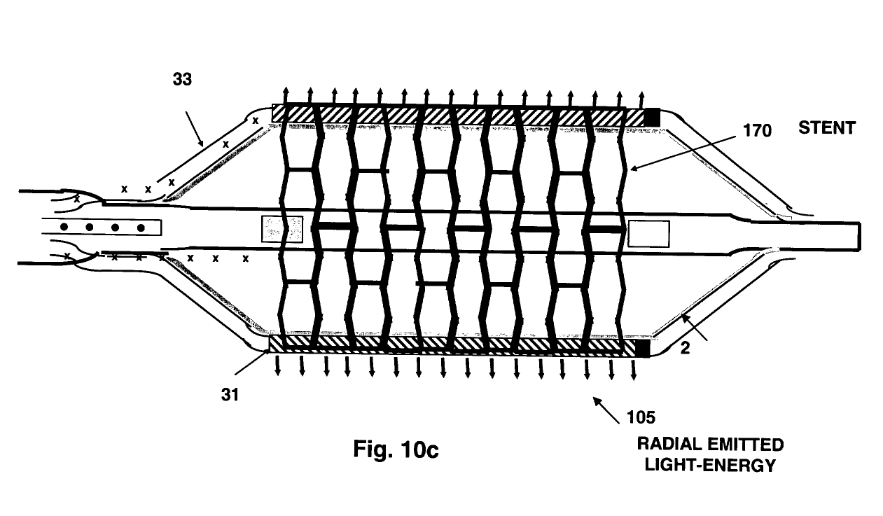

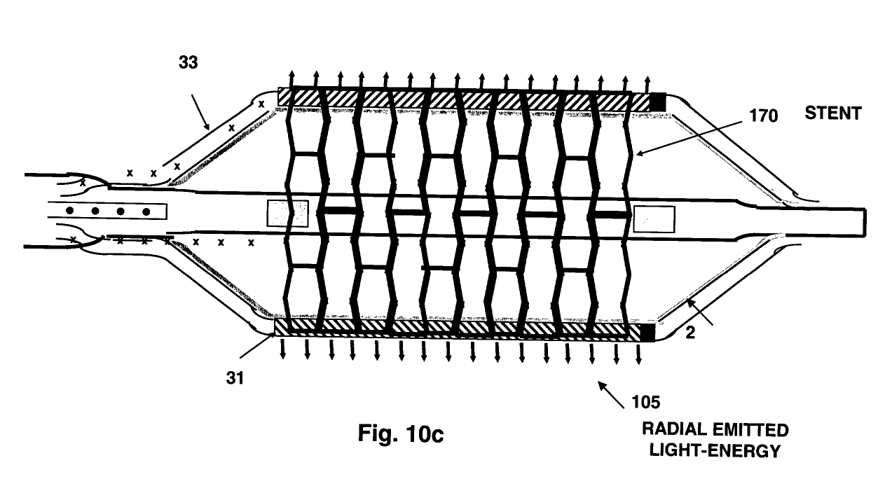

The following sections describe alternative methods of routing and mounting externally attached optical fibers 3 onto balloon body 2. Reference is now being made to FIG. 10a. According to the embodiment, illustrated in FIG. 1 to 9 and as detailed above, optical fiber proximal section 33 is routed inside the catheter wall 4 and exits through a bore in catheter wall intermediary section 42 or the externally attached optical fibers 3 are routed externally along the catheter wall 4. FIG. 10a illustrates an alternative design and apparatus for routing externally attached optical fibers 3. As illustrated in FIG. 10a, externally attached optical fibers 3 are routed through the catheter wall intermediary section 42 and exit tube-like catheter wall 4 through distal catheter member 41. In this alternative embodiment, intermediary optical guide-member 51, if used, is continued till distal catheter member 41 that includes distal optical connector 52. Extension member 34, can optionally be used due to mechanical considerations and can be externally connected to catheter wall intermediary section 42. Preferably, optical fiber proximal section 33 has a “spare” part that can glide through distal catheter member 41 while maintaining light-energy transmission capability, and enables proper wrapping and re-wrapping during inflation/deflation of balloon body 2.

Reference is now made to FIG. 10b. The set of optical fibers 3 are connected in a cooperative structure by means of at least one elastic fiber bonding element 36. Similar to embodiment described in previous sections each optical fiber 3 has at its distal end a light-energy emitting section 31 ending in a totally reflecting element 32 and an optional extension member 34. Optionally, extension members 34 are attached together in a distal connecting member 35 that has the structure and properties of a regular guide-wire tip. As illustrated, prior to the procedure the set of optical fibers 3 is separately packaged and is not yet mounted onto dilatation balloon catheter 1 and balloon body 2. Just before the procedure the user stretches bonding element(s) 36 and slides set of optical fibers 3 onto balloon body 2. The elasticity of fiber bonding element 36 (one or more) enables to slide the structure built from set of optical fibers 3 onto dilatation balloon catheter 1, such that light energy emitting section 31 is positioned and firmly attached onto balloon body 2, and particularly on the non-tapered part of balloon body 2. After the mounting of the optical fibers 3 onto balloon body 2, it is possible to operate dilatation balloon catheter 1 and externally attached set of optical fibers 3 as described in relation to the embodiments illustrated in FIG. 1 to 9. Fiber bonding elastic element(s) 36 also ensures that externally attached optical fibers 3 remain: attached to balloon body 2 during the operation, and particularly that they re-wrap properly (together with balloon body 2) after balloon body 2 is deflated. The design illustrated in FIG. 10b can facilitate the attachment of external optical fibers 3 to virtually any dilatation balloon catheter 1.

Reference is now made to FIG. 10c. Dilatation balloon catheter 1 including externally attached optical fibers 3 is used for delivering a stent 170 to treat a stenosed lesion. Primary stenting is currently limited by the risk of the stent being stuck in cases of unsuccessful dilatation. This requires as a first step to dilate the stenosis by using a dilatation balloon catheter and only after the successful dilatation to bring the stent and position it in order to secure the lesion. Based on the capability of dilating hard to expand: lesions dilatation balloon catheter 1 with externally attached optical fibers 3 can be used as a delivery system for primary stenting diminishing the risk of an unsuccessful dilatation. According to a preferred option stent 170 is a regular stent and the radial emitted light-energy due to the selected wavelength and intensity is not causing any damage to the stent, and it is not altering its properties. This is due to the fact that, for example short light pulses with energy levels capable of heating or ablating adjacent-tissue will not affect stent 170. According to one possible design, the attachment points of stent 170 to externally attached optical fibers 3 are thermally shielded avoiding thermal diffusivity due to thermal conductivity of stent 170. According to an alternate design, stent 170 has a special structure ensuring its bonding to balloon body 2 during its transition. According to still additional options, stent 170 is attached to balloon body 2 by bonding elements that release stent 170 from balloon body 2 after emitting light-energy through externally attached optical fibers 3.

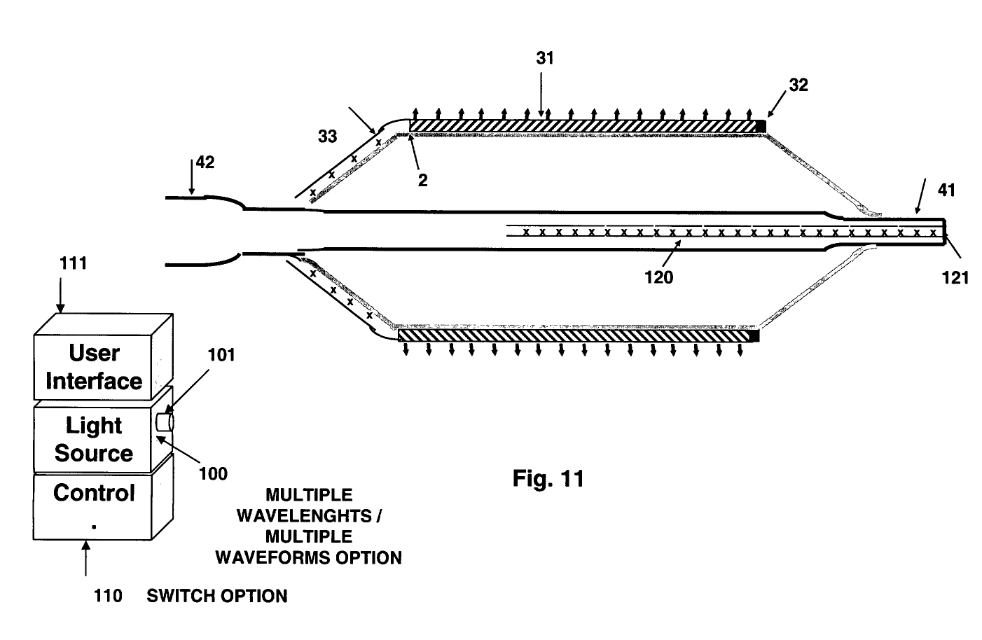

Reference is now made to FIG. 11 that illustrates a dilatation balloon catheter 1 that includes a set of externally attached optical fibers 3 and also one or more internal forward emitting light-delivery elements 120. The functionality of externally attached optical fibers 3 is as described in the embodiments illustrated in FIG. 1 to 13. The internal light-delivery element 120 has the role to assist in crossing dilatation balloon catheter 11 through partial occlusions or chronic total occlusions. According to a preferred embodiment light-delivery element 120 is a fiber bundle being connected at their proximal side to light-source 100 and having a fiber distal end 121 capable of emitting forward light-energy. Fiber distal end 121 is passed through distal catheter member 41. Light-source 100 has an output optical design enabling the user to select between the usage of the forward emitting light-delivery element 120 for crossing occlusions and between usage of externally attached optical fibers 3 for dilating the occlusion (once the occlusion was first crossed).

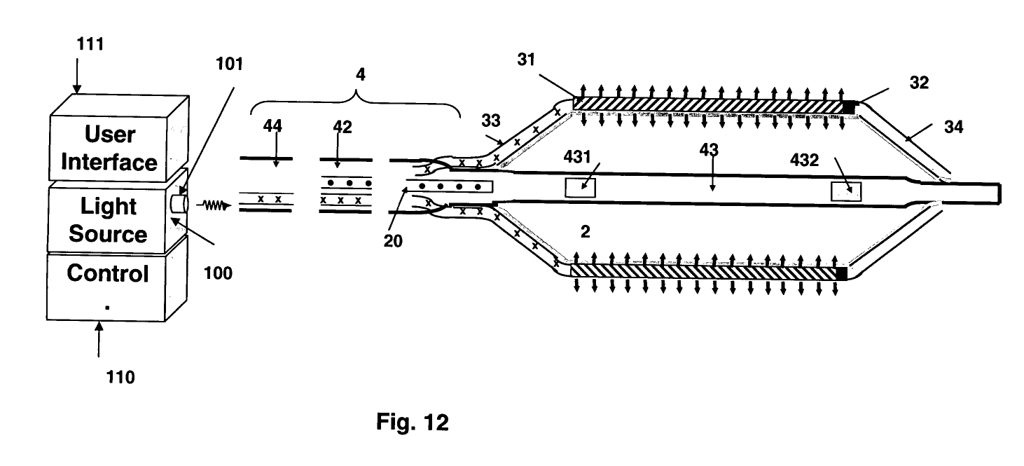

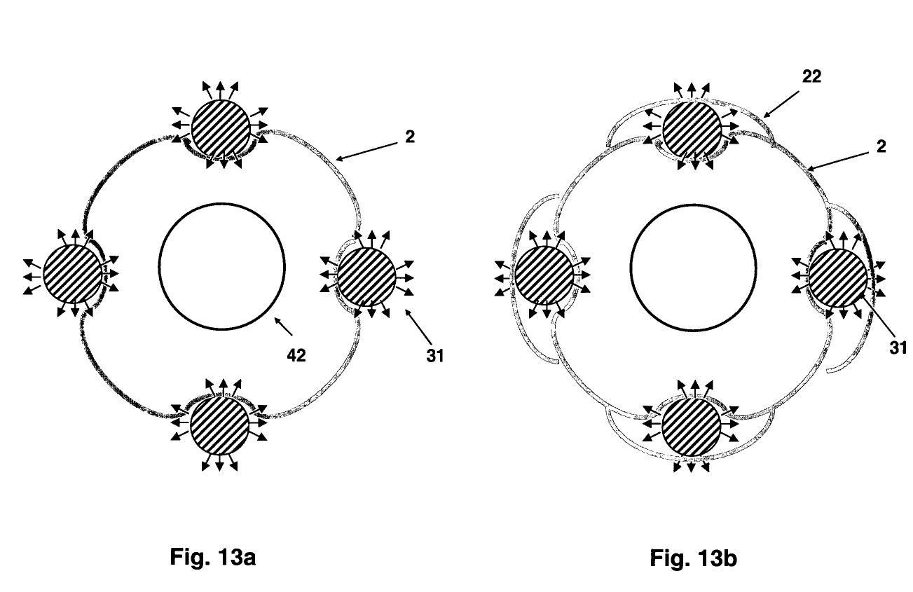

Reference is now being made to FIG. 12, FIG. 13a and FIG. 13b illustrating a second embodiment of the current invention. This embodiment describes apparatus and methods for locally activation of light sensitive drugs by means of light-delivery elements attached to a dilatation balloon catheter 1. Set of externally attached optical fibers 3 is connected to light source 100 as described in the embodiments illustrated in FIG. 1 to 10. In order to achieve the required interventional and therapeutic effect each externally attached optical fiber 3 has a light-energy emitting section 31 capable of emitting radial light energy 105 preferably in the form of a diffused light wave as further described. As illustrated, radial light energy 105 is preferably emitted 360 degrees directly towards blood vessel wall 150 and/or through balloon body 2 that can be made transparent to the specific wavelength(s) emitted by light-energy emitting section 31. Balloon body 2 can also have areas that are opaque to the specific wavelength(s) emitted by light-energy emitting section 31 in order to ensure that the activation of the drug is prevented outside the required area. Optionally, as illustrated in FIG. 13b, balloon body 2 may include an external membrane 22 over the light-energy emitting section 31. Usage of optional external membrane 22 can further spread the light-energy emitted by light-energy emitting section 31.

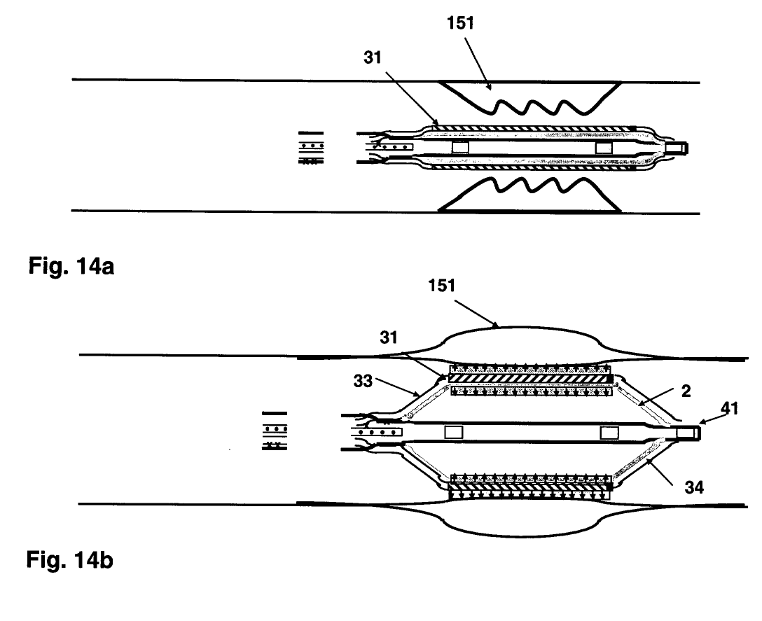

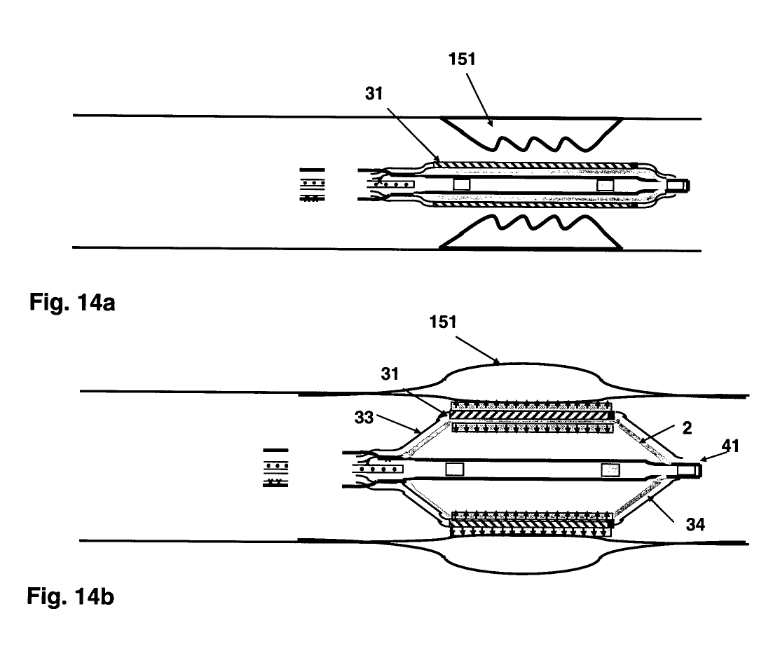

Reference is now made to FIG. 14a, 14b that illustrate the steps in using dilatation balloon catheter 1 illustrated in FIG. 12. As a first step, catheter 1 including balloon body 2 is maneuvered through the vessel and positioned in the area of interest, for example the stenosed vessel segment including the atherosclerotic lesion. Once balloon body 2 is in position, the second step includes the activation of light source 100 in order to achieve an optimal energy level. Light-energy emitting section 31 can emit radial light-energy before, during and after inflating balloon body 2 according to any required workflow. The emitted light energy activates the light sensitive drug. The special structure of light-energy emitting section 31 ensures delivery of radial light energy 105 to blood vessel wall 150 and atherosclerotic plaque 151.

The light activated drug can be delivered to the required blood vessel section by various methods. According to a first preferred method, the light activated drugs are delivered by the balloon catheter itself. Various schemes are readily available for such drug delivery, such as Microporous Balloon manufactured by Cordis, the Dispatch Delivery catheter by Scimed, Infusion Sleeve by LocalMed Inc., Hydrogel balloon by Boston Scientific, etc. Each of the above mentioned balloon catheters can be used in connection to the current invention with the proper adjustments necessary for adding the external light delivery means attached to balloon body 2.

According to; an alternative method the light activated drugs are delivered by injections in the area of interest, or unselectively through out the entire blood vessel wall.

Various drugs/agents can be used in connection to PDT. The group of relevant drugs/agents includes, but it is not limited to streptokinase, urokinase, heparin, enoxaparine, etc.

According to a preferred embodiment, the same set of externally attached optical fibers 3 with light energy emitting section 31 can assist the dilatation process by creating microscopic longitudinal cuts through the induced opto-mechanical effects described in connection to the embodiments illustrated in FIGS. 1-10, and also can be used for activating photosensitive drugs. In order to achieve optimal clinical results, two different workflows can be implemented.

According to a first preferred method and apparatus the wavelength used for creating the microscopic light-energy opto-mechanical cuts is different than the wavelength used for activating the light sensitive drug. In this case the balloon is first inflated and only then the drug is activated.

According to at second method and apparatus the same wavelength is used for creating the microscopic light-energy opto-mechanical cuts and for activating the light sensitive drug. In this case the drug is activated during or after the inflation process, according to the drug delivery method.

Taking, for example, a light-source capable of generating wavelength(s) of 0.308 microns and also of 1.053 microns, the following methodology can be implemented (similar methodologies can be implemented at other wavelengths). After positioning the dilatation balloon in the stenotic area to be dilated the light source is activated at 308 microns. At this wavelength for most types of tissue the absorption coefficient is high, (i.e. the penetration depth is very small), and the radial confined light-energy emitted by externally attached light-energy emitting sections 31 combined with their pressure create microscopic longitudinal cuts or “cracks” in the atherosclerotic plaque 15. After the successful dilatation of the stenotic lesion light-source 100 emits low intensity light-energy at 1.053 microns in order to stimulate drug delivery and/or drug activation that is useful for example in preventing restenosis. Optionally, the emission at 1.053 microns can be activated in parallel to the dilatation process.

The advantage of using externally attached optical fibers 3 emitting light energy for activation of light sensitive drugs in vascular applications and similar clinical applications such as treatment of stenoses in the biliary system the urinary system or gastrointestinal system, resides in the following aspects that are advantageous for such interventions:
The area where the drug is activated is easily controlled.
Activation of light sensitive drugs is easier and safer to perform;
The same dilatation balloon can be used for multiple therapy tasks including controlled inflation integrated with microscopic light-energy opto-mechanical cuts as described in the first preferred embodiment of the current invention

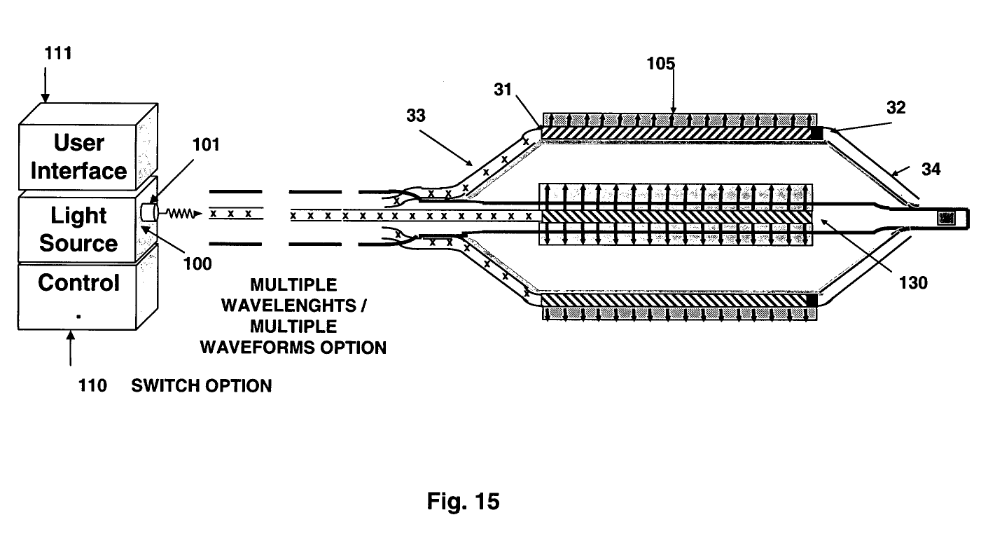

Reference is now made to FIG. 15 illustrating an alternative optical design facilitating the dual therapy effects described in the previous sections in connection to FIGS. 12-13. Dilatation balloon catheter 1 includes at least one externally attached optical fiber 3 with a light energy emitting sections 31 capable of emitting radial light energy 105 and also includes at least one internal radial emitting light-delivery element 130. In this embodiment, the externally attached optical fibers 3 have the same functionality as described in the embodiments illustrated in FIG. 1 to 10. The internal radial emitting optical element 130 has the role of stimulating drug delivery and/or drug activation. Light-source 100 and light-source optical output member 101 enable the user to: select between using externally attached optical fibers 3 for dilating the occlusion (once the occlusion was first crossed) and between using internal radial emitting optical element 130 for photodynamic therapy. Optionally, light-source 100 and light-source optical output member 101 are capable of supporting parallel emission through both externally attached optical fibers 3 and through internal radial emitting optical element 130 at the same wavelength or at separate wavelengths as described in previous sections. Still optionally, light-source 100 and light-source optical output member 101 are capable of supporting by multiplexing or in parallel emission through forward emitting light-delivery elements 120 (as illustrated in FIG. 11 and described in previous sections), emission through externally attached optical fibers 3 for dilating the occlusion (once the occlusion was first crossed) and emission through internal radial emitting optical element 130 for photodynamic therapy.

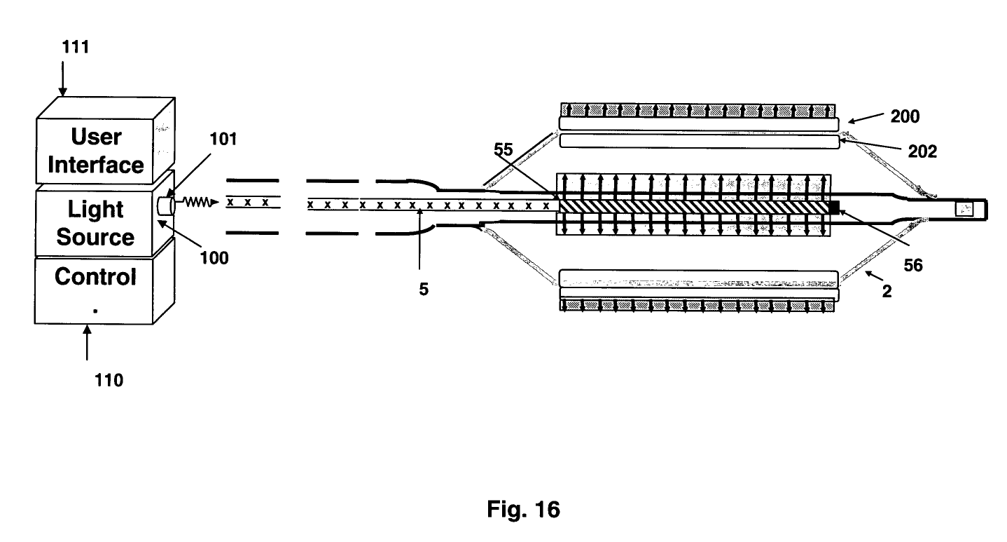

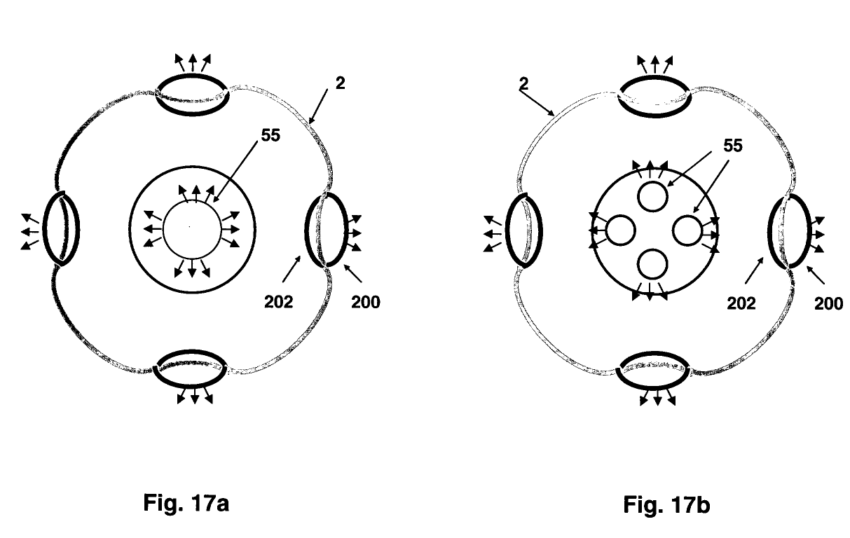

Reference is now made to FIG. 16, FIG. 17a and FIG. 17b that illustrate an alternative design and apparatus for a dilatation balloon catheter capable of dilating stenotic vessels at lower pressures with minimal induced damage to the vessel wall. With respect to FIG. 17a, a dilatation balloon catheter 1 includes a light energy guide system 5 with an optical inter-balloon element 55 capable of emitting radial light-energy. The distal part of optical inter-balloon element 55 ends in totally reflecting element 56. The optical design described in connection to light energy emitting section 31 is preferably used for optical inter-balloon element 55 in order to ensure uniformity and high efficiency. The diameter of optical inter-balloon element 55 is preferably between 200-300 microns capable of transmitting higher energy levels. Alternate diameters can be used according to the size of the vessel and the clinical application.

According to a first preferred apparatus, a set of externally attached optical elements 200 are attached or embedded in balloon body 2 and are capable of transmitting radial confined light-energy 105 from optical inter-balloon element 55 to blood vessel wall, 150 and stenotic plaque 151. Externally attached optical elements 200 have a longitudinal shape spanning the entire balloon section (the non-tapered) section used for dilating the stenotic vessel, and are preferably equally spread on the perimeter of the balloon body 2. Externally attached optical elements 200 can be designed to act as a light-focusing element or can be designed to be only an optical window for transferring the light-energy to the adjacent tissue. Externally attached optical elements 200 have a firm, yet flexible structure ensuring that during the inflation of balloon body 2 they exert an adequate pressure on the heated/ablated/evaporated/bond-weekend tissue adjacent to them. The structure of externally attached optical elements 200 increases the endoluminal maneuverability of dilatation balloon catheter 1.

Additionally, an optional intra-balloon focusing element 202 can be used to focus the radial light energy emitted by optical inter-balloon element 55 on the externally attached optical element 200 and/or a confined tissue volume adjacent to them. According to further aspects of the current invention intra-balloon focusing elements 202 expand or change their angle as the balloon expands, in order to ensure a fixed distance from balloon body 2 and externally attached optical elements 200, and to ensure that radial confined light energy is delivered to tissue adjacent to attached light focusing elements 200 regardless of the inflation level of the balloon body 2. Other optical methods for focusing the radial emission through externally attached optical elements 200 may include angulations of intra-balloon focusing element 202 according to the inflation of balloon body 2. Optionally, externally attached optical elements 200 and/or intra-balloon focusing elements 202 are part of balloon body 2. Externally attached optical elements 200 and intra-balloon focusing element 202 are manufactured form materials and have an optical design in accordance to the selected wavelength and intensity.

According to alternate optical design, optical inter-balloon element 55 has a special optical design ensuring that its radial emission is focused on externally attached optical elements 200. According to further aspects of the current invention, balloon body 2 can be coated in certain areas to reflect the radial light-energy emitted by inter-balloon section 54 and provide additional light-focusing on the externally attached optical element 200. The focusing effect may also be achieved by varying the width and materials of balloon body 2 in certain areas.

FIG. 17b illustrates an alternative apparatus where light energy guide system 5 and optical guide inter-balloon section 55 includes several optical fibers each emitting radial light-energy. Optionally, these optical fibers can optionally expand as the balloon body 2 is inflated maintaining a fixed distance from externally attached optical elements 200 regardless of the inflation level of balloon body 2.

During a Percutaneous Transluminal Angioplasty procedure dilatation balloon catheter 11 is connected to a light-source 100 and operated similarly to the operation of dilatation balloon catheter 1 described in above embodiments that are illustrated in FIG. 1 to 13. Externally attached optical elements 200, facilitate a smooth inflation process due to an optimal combination of optical and mechanical effects that create longitudinal microscopic cuts or bond-weaken segments in the stenotic plaque 151. Similar to the described functionality of dilatation balloon catheter 1 and light energy emitting section 31, externally attached optical elements 200 deliver confined longitudinally uniform light-energy to a microscopic volume of adjacent tissue and at the same time due to its firm structure and shape exerts mechanical pressure on the same volume of adjacent tissue. Once a required inflation level is achieved the light-delivery is stopped in order to ensure that no unwanted damage is induced on vessel wall 150.

Selective activation can be implemented when using externally attached optical elements 200 pending that optical guide inter-balloon section 55 is designed to enable selecting the emitting section, for example as illustrated in FIG. 15b.

Optical inter-balloon element 55 and externally attached optical elements 200 can also be used for activating light sensitive drugs as described in previous embodiments. In this case, according to a first preferred implementation the same wavelength is used for creating the microscopic opto-mechanical cuts and also for the drug activation. According to an alternate implementation one single/set of wavelengths is used for creating the microscopic opto-mechanical cuts and another single/set of wavelengths is used for the drug activation. In the later case balloon body 2 can be coated in certain areas as to reflect the light-energy at the wavelength(s) used for creating the microscopic cuts and at the same time being transparent for the wavelength(s) used for drug activation.

Optionally, light-source 100 and light-source optical output member 101 are capable of supporting by multiplexing or in parallel emission through forward emitting light-delivery elements 120 (as illustrated in FIG. 10d and described in previous sections) and emission through optical inter-balloon element 55 for dilating the occlusion (once the occlusion was first crossed) and for photodynamic therapy.

The various opto-mechanical designs, the various apparatus and methods described in relation to laser source 100 and control unit 110, the various designs for designing balloon body 2 and the bonding of external optical elements to balloon body 2, and other variations described in connection to the usage of externally attached optical fiber 3 are equally applicable to the usage of optical inter-balloon element 55 and externally attached optical elements 200.

Numerous modifications and variations of the present invention are possible in light of the above teaching. Particularly, the dilatation balloon catheter and methods disclosed in the current invention can be used in PTA procedures in the vascular system, urinary system, biliary system or gastrointestinal system. The dilatation balloon catheters and methods disclosed in the current invention can be used in PTA procedures in humans or mammalian animals. Although the invention has been described and illustrated in detail, the above descriptions and illustrations have been offered for illustrative purposes only, and is not intended to limit the scope of the invention of this application.

1. A dilatation balloon catheter comprising:
a balloon body for dilating stenotic lesions;
at least one optical fiber including a optical fiber distal segment externally attached on the perimeter of said balloon body;
where said at least one optical fiber can be connected to a light source and said optical fiber distal segment is capable of receiving light energy from said light source and emitting radial light energy to an adjacent tissue volume with high optical efficiency;
where said radial emitted light energy is substantially uniform along said optical fiber distal segment and the light-tissue interaction depth enables to interact with a very confined tissue volume proximal to said distal segment;
where said optical fiber distal segment has a firm structure that it enables during the inflation of said balloon body to exert pressure exactly on said confined adjacent tissue volume that interacted with said radial emitted light-energy;
where said radial emitted light energy and said exerted pressure create microscopic cracked or cut or bond-weaken segments in the stenotic tissue;
and said microscopic cracked or cut or bond-weaken segments in the stenotic tissue facilitate the dilatation of said stenotic lesions by means of said dilation balloon catheter.

2. A dilatation balloon catheter according to claim 1 where said dilatation balloon catheter can be inflated and deflated one or more times for therapy purposes, and said optical fiber distal segment is capable of remaining attached to said balloon body regardless of the inflation or deflation position of said basic balloon body.

3. A dilatation balloon catheter according to claim 1 where said at least one optical fiber is more than one optical fiber.

4. A dilatation balloon catheter according to claim 1 where said optical fiber distal segment can have any length of up to 10 cm.

5. A dilatation balloon catheter according to claim 1 where said microscopic cracked or cut or bond-weaken segments in the stenotic tissue are achieved due to said radial emitted light energy.

6. A dilatation balloon catheter according to claim 1 where said microscopic cracked or cut or bond-weaken segments in the stenotic tissue are achieved due to a combination of said radial emitted light energy and of said exerted pressure.

7. A dilatation balloon catheter according to claim 1 where said optical fiber distal segment is manufactured from a flexible and firm material ensuring enhanced endoluminal maneuvering capabilities through tortuous vessels and other stenoses or obstructions.

8. A dilatation balloon catheter according to claim 1 where the material of said balloon body is matched to the effect induced by said optical fiber distal segment in said stenotic tissue.

9. A dilatation balloon catheter according to claim 1 where the inflation rate of said balloon body is matched to the effect induced by said optical fiber distal segment in said stenotic tissue.

10. A dilatation balloon catheter according to claim 1 where said clinical endoluminal intervention can be related to any of the following group: vascular system, biliary system, urinary system or gastrointestinal system.

11. Clinical invasive intervention according to claim 1 where said clinical endoluminal intervention is performed on a human or on a mammalian animal.

12. An angioplasty method for dilating a stenotic lesion by means of a dilatation balloon catheter including a balloon body and at least one optical fiber including an optical fiber distal segment that is externally attached on the perimeter of said balloon body, said method including the steps of:
a first step of positioning said balloon body with said externally attached optical fiber distal segment against said stenotic lesion;
a second step of activating a light source and transferring said light energy to said optical fiber distal segment, where said optical fiber distal segment is capable to emit radial light-energy to an adjacent tissue volume with high optical efficiency;
where said radial emitted light energy is substantially uniform along said optical fiber distal segment and the light-tissue interaction depth enables to interact with a very confined tissue volume proximal to said distal segment;
a third step of inflating said balloon body with said optical fiber distal segment, where said optical fiber distal segment has a firm structure that exerts pressure exactly on said confined adjacent tissue volume that interacts with said radial emitted light-energy;
where said radial emitted light energy and said exerted pressure create microscopic cracked or cut or bond-weaken segments in the stenotic tissue;
and where said microscopic cracked or cut or bond-weaken segments in the stenotic tissue facilitate the dilatation of said stenotic lesions by means of said dilation balloon catheter.

13. A percutaneous transluminal angioplasty method according to claim 12 where externally attached light energy means are manufactured from a flexible and firm material ensuring enhanced endoluminal maneuvering capabilities through tortuous vessels and other stenoses or obstructions.

14. A percutaneous transluminal angioplasty method according to claim 12 where said clinical invasive intervention can be related to any of the following group: vascular system, biliary system, urinary system or gastrointestinal system.

15. Clinical invasive intervention according to claim 14 where said clinical endoluminal intervention is performed on a human or on a mammalian animal.
